(19) United States
(12) Patent Application Publication
(10) Pub. No.: US 2025/0352279 A1
(43) Pub. Date: Nov. 20, 2025

(54) Systems And Methods For Implementing Adjunct Technology To Facilitate A Surgical Procedure

(71) Applicant: Stryker Corporation, Portage, MI (US)

(72) Inventors: Martin Griffin, Orange, CA (US); Ross Nave, Kalamazoo, MI (US); Slaven Sutalo, Byron City, MI (US)

(73) Assignee: Stryker Corporation, Portage, MI (US)

(21) Appl. No.: 19/203,880

(22) Filed: May 9, 2025

Related U.S. Application Data
(60) Provisional application No. 63/647,971, filed on May 15, 2024.

(51) Int. Cl. A61B 34/00; A61B 90/00; G16H 40/67

(52) U.S. Cl. CPC A61B 34/25; G16H 40/67; A61B 2090/0804; A61B 2560/0493

(57) ABSTRACT

A method of facilitating a surgical procedure includes receiving a first input including a first surgical article type and a first quantity of the first surgical article type for surgical articles that have been counted in to the procedure, displaying an electronic count including the first surgical article type and the first quantity of the first surgical article type, and receiving a subsequent input to alter the count. The method further includes capturing, with a microphone, speech including a verbal count for the surgical articles that have been counted in to the procedure, converting the verbal count into machine-encoded values including a second surgical article type and a second quantity, and comparing the electronic count to the machine-encoded values to identify a discrepancy. The method further includes displaying the identified discrepancy.

### CROSS REFERENCE TO RELATED APPLICATIONS

This application claims the benefit of U.S. Provisional Patent Application No. 63/647,971 filed on May 15, 2024, which is hereby incorporated by reference.

### BACKGROUND

Prior to and during a surgical procedure, it is imperative that clinical staff accurately perform all procedure protocols, for example, verifying patient identification and executing item counts. In particular, the clinical staff needs to ensure an accurate inventory of surgical sponges that are “counted in” to be used in the surgical field, and those “counted out” before conclusion of the surgical procedure to avoid a surgical sponge being inadvertently left in an inappropriate location. Often, the procedure protocols are performed manually, and outputs recorded on a whiteboard that is located in the operating room. For example, the clinical staff may count the surgical sponges by hand, which is prone to human error, and write the manual count on the whiteboard, which is prone to illegibility. More recently, surgical sponges have been tagged with radiopaque markers, barcodes, and/or wireless transponders, such as radiofrequency identification (RFID) tags, to facilitate electronically counting the surgical sponges wirelessly with a data reader. One exemplary manner by which this is implemented is disclosed in commonly-owned United States Patent Publication No. 2022/0246288, published Aug. 4, 2022, the entire contents being hereby incorporated by reference. Such adjunct technology addresses certain shortcomings with manual counting, yet it would be desirable to further integrate the technology into the various procedural protocols of the surgical procedure.

### SUMMARY

According to a first aspect, a computer-implemented method of facilitating a surgical procedure with a surgical management system including a display device, a user interface, one or more microphones, and one or more processors is provided. The method includes receiving, with the user interface, a first input including a first surgical article type and a first quantity of the first surgical article type for a plurality of surgical articles that have been counted in to the surgical procedure and displaying, on the display, an electronic count including the first surgical article type and the first quantity of the first surgical article type. The method further includes receiving, on the user interface, one or more subsequent inputs to alter the electronic count and displaying, on the display, the altered electronic count. The method further includes capturing, with the one or more microphones, audible speech including a confirming verbal count for the plurality of surgical articles that have been counted in to the surgical procedure and converting, with the one or more processors, the confirming verbal count into machine-encoded values including a second surgical article type and a second quantity of the second surgical article type. The method further includes comparing, with the one or more processors, the altered electronic count to the machine-encoded values to identify a discrepancy between the altered electronic count and the machine-encoded values and displaying, upon the display device, the identified discrepancy between the altered electronic count and the machine-encoded values.

According to a second aspect, a computer-implemented method of facilitating a surgical procedure with a surgical management system including a display device, a user interface, one or more microphones, and one or more processors is provided. The method includes receiving, with the user interface, a first input including a first list of a plurality of surgical article types and a first count of a quantity of each of the plurality of surgical article types for a plurality of surgical articles that have been counted in to the surgical procedure and displaying, on the display, an electronic count of the first list of the plurality of surgical article types and the first count of the quantity of each of the plurality of surgical article types for the plurality of surgical articles that have been counted in to the surgical procedure. The method further includes receiving, on the user interface, one or more subsequent inputs to alter the electronic count and displaying, on the display, the altered electronic count. The method further includes capturing, with the one or more microphones, audible speech including a confirming verbal count for the plurality of surgical articles that have been counted in to the surgical procedure and converting, with the one or more processors, the confirming verbal count into machine-encoded values including a second list of the plurality of surgical article types and a second count of the quantity of each of the plurality of surgical article types that have been counted in to the surgical procedure. The method further includes comparing, with the one or more processors, the altered electronic count to the machine-encoded values to identify a discrepancy between the altered electronic count and the machine-encoded values and displaying, upon the display device, the identified discrepancy between the altered electronic count and the machine-encoded values.

According to a third aspect, a computer-implemented method of facilitating a surgical procedure with a surgical management system including a display device, a user interface, one or more microphones, and one or more processors is provided. The method includes, within the one or more processors, training a neural network by iteratively receiving an auditory input including one of a plurality of training verbal inputs including a training list of a plurality of surgical article types and a training count of a quantity of each of the plurality of surgical article types, the plurality of training verbal counts being configured to simulate a user counting in a plurality of surgical articles into the surgical procedure. Training the neural network further includes recognizing data related to one of the plurality of surgical article types or the quantity of each of the plurality of surgical article types from the received one of the plurality of training verbal inputs. Training the neural network further includes, based upon the recognized data, generating a training list result of the plurality of surgical article types and a training quantity result of each of the plurality of surgical articles types and scoring the training list result and the training quantity result based upon details of the one of the plurality of training verbal inputs. Training the neural network further includes correcting a next iteration of recognizing the data based upon the scoring. The method further includes receiving, with the user interface, a first input including a first list of a plurality of surgical article types and a first count of a quantity of each of the plurality of surgical article types for a plurality of surgical articles that have been counted in to the surgical procedure. The method further includes displaying, on the display, an electronic count of the first list of the plurality of surgical article types and the first count of the quantity of each of the plurality of surgical article types for the plurality of surgical articles that have been counted in to the surgical procedure and receiving, on the user interface, one or more subsequent inputs to alter the electronic count. The method further includes displaying, on the display, the altered electronic count and capturing, with the one or more microphones, audible speech including a confirming verbal count for the plurality of surgical articles that have been counted in to the surgical procedure. The method further includes utilizing, within the one or more processors, the trained neural network to process the audible speech and generate a recognized list of the plurality of surgical article types and a recognized count of the quantity of each of the plurality of surgical article types and comparing, with the one or more processors, the altered electronic count to the recognized list and the recognized count to identify a discrepancy between the confirming verbal count and the altered electronic count. The method further includes displaying, upon the display device, the identified discrepancy.

In one implementation, comparing the altered electronic count to the machine-encoded values includes comparing the first surgical article type to the second surgical article type and comparing the first quantity of the first surgical article type to the second quantity of the second surgical article type.

In one implementation, the method further includes receiving, with the user interface, a count out input including a counted-out quantity of the first surgical article type for a plurality of surgical articles that have been counted out of the surgical procedure, updating the electronic count based upon the counted-out quantity of the first surgical article type, and displaying, on the display, the updated electronic count of the first surgical article type and the first quantity of the first surgical article type.

In one implementation, the first input further includes a first list of a plurality of surgical article types and a first count of a quantity of each of the plurality of surgical article types for the plurality of surgical articles that have been counted in to the surgical procedure. In one implementation, the electronic count further includes the first list of the plurality of surgical article types and the first count of the quantity of each of the plurality of surgical article types for the plurality of surgical articles that have been counted in to the surgical procedure. In one implementation, the machine-encoded values further include a second list of the plurality of surgical article types and a second count of the quantity of each of the plurality of surgical article types that have been counted in to the surgical procedure.

In one implementation, the method further includes receiving, with the user interface, a count out input including the first list of the plurality of surgical article types and a count of a counted-out quantity of each the plurality of surgical article types for a plurality of surgical articles that have been counted out of the surgical procedure, updating the electronic count based upon the count out input, and displaying, on the display, the updated electronic count.

In one implementation, the method further includes prompting the confirming verbal count prior to authorizing a portion of the surgical procedure to commence.

In one implementation, the confirming verbal count is a first confirming verbal count, and the machine-encoded values are a first set of machine-encoded values. In one implementation, the method further includes capturing, with the one or more microphones, additional audible speech including a second confirming verbal count for the plurality of surgical articles that have been counted in to the surgical procedure and converting, with the one or more processors, the second confirming verbal count into a second set of machine-encoded values including a third surgical article type and a third quantity of the third surgical article type that have been counted in to the surgical procedure. In one implementation, the method further includes further comparing, with the one or more processors, the second set of machine-encoded values to the altered electronic count and to the machine-encoded values and displaying, upon the display device, a second discrepancy either between the second set of machine-encoded values and the altered electronic count or between the second set of machine-encoded values and the first set of machine-encoded values.

In one implementation, the method further includes registering a user, identifying the registered user as entering the one or more subsequent inputs, and displaying, upon the display, an indication that the registered user entered the one or more subsequent inputs.

In one implementation, the method further includes registering a plurality of users authorized to alter the electronic count and receiving a plurality of subsequent inputs to alter the electronic count. In one implementation, the method further includes, for each of the plurality of subsequent inputs, identifying one of the plurality of users as being a respective user that entered the subsequent input and displaying, on the display, for each of the plurality of subsequent inputs, an indication identifying the user that entered the subsequent input.

In one implementation, the user interface is a touch screen device. In one implementation, the method further includes, upon the touch screen device, displaying the first surgical article type for a plurality of surgical articles that have been counted in to the surgical procedure and displaying, adjacent to the displayed first surgical article type, a first button indicating an increase and a second button indicating a decrease. In one implementation, the method further includes monitoring a user indicating by touch to one of the first button or the second button and receiving the first input or one of the one or more subsequent inputs based upon the monitored indicating.

In one implementation, the method further includes determining a number of users that entered the one or more subsequent inputs and comparing the determined number of users that entered the subsequent inputs to a threshold number of users. In one implementation, the method further includes generating an alert when the comparing indicates that the determined number of users that entered the subsequent inputs exceeds the threshold number of users, wherein the alert is configured to raise awareness that an increased risk of miscounting is indicated due to the determined number of users that entered the subsequent inputs.

In one implementation, the method further includes receiving, with the user interface, a count out input including the first list of the plurality of surgical article types and a count of a counted-out quantity of each the plurality of surgical article types for a plurality of surgical articles that have been counted out of the surgical procedure, updating the electronic count based upon the count out input, and displaying, on the display, the updated electronic count.

In one implementation, comparing the altered electronic count to the machine-encoded values includes comparing the first list of the plurality of surgical article types to the second list of the plurality of surgical article types and comparing the first count of the quantity of each of the plurality of surgical article types to the second count of the quantity of each of the plurality of surgical article types.

### BRIEF DESCRIPTION OF THE DRAWINGS

FIG. 1A is a perspective view of a surgical system. The surgical system includes a surgical sponge management system.

FIG. 1B is a perspective view of the surgical sponge management system of FIG. 1A. The surgical sponge management system includes a display interface.

FIG. 2 is a view of the display interface of FIG. 1B, illustrating a plurality of selectable procedures that are scheduled for a particular operating room.

FIG. 3 is a view of the display interface of FIG. 1B illustrating a dashboard view configured to enable one to manage the surgical sponge management system throughout a surgical procedure. An option to review details regarding the patient is currently selected.

FIG. 4 is a view of the display interface of FIG. 1B illustrating a patient summary page. Details of the patient are listed and a status of a patient consent form is provided.

FIG. 5 is a view of the display interface of FIG. 1B illustrating an exemplary patient consent form. A signature field which may be presented to patient for touch screen signature.

FIG. 6 is a view of the display interface of FIG. 1B illustrating the patient summary page of FIG. 4. The status of the patient consent form is updated to reflect the signed document.

FIG. 7 is a view of the display interface of FIG. 1B, illustrating the dashboard view of FIG. 3. An option to review checklists for the surgical procedure is selected.

FIG. 8 is a view of the display interface of FIG. 1B illustrating a checklist summary page providing an ability to select between various checklists that are available for the surgical procedure.

FIG. 9 is a view of the display interface of FIG. 1B illustrating an exemplary sign-in checklist.

FIG. 10 is a view of the display interface of FIG. 1B illustrating an exemplary timeout checklist.

FIG. 11 is a view of the display interface of FIG. 1B illustrating an exemplary sign-out checklist.

FIG. 12 is a view of the primary display screen of FIG. 1A illustrating a surgical procedure summary screen.

FIG. 13 is a view of the display interface of FIG. 1B illustrating the dashboard view of FIG. 3. An option to enter a surgical article count is selected.

FIG. 14 is a view of the display interface of FIG. 1B illustrating a new tablet count management screen.

FIG. 15 is a view of the display interface of FIG. 1B illustrating the new tablet count management screen including a staff ID registration window to correlate new count entries with a particular medical professional.

FIG. 16 is a view of the display interface of FIG. 1B illustrating a screen for adding a new surgical article type to a count.

FIG. 17 is a view of the display interface of FIG. 1B illustrating a count in tablet entry screen, enabling a user to add surgical articles to a count with the tablet during a count in process.

FIG. 18 is a view of the display interface of FIG. 1B illustrating the count in tablet entry screen of FIG. 17. A user has entered ten 2×2 sponge pads into the surgical procedure.

FIG. 19 is a view of the display interface of FIG. 1B illustrating a count in summary page reflecting the exemplary ten 2×2 sponge pads that were entered into the count with the count in tablet entry screen of FIG. 18.

FIG. 20 is a view of the primary display screen of FIG. 1A illustrating the surgical procedure summary screen of FIG. 12. The summary screen reflects the exemplary ten 2×2 sponge pads that have been entered into the procedure.

FIG. 21 is a view of the display interface of FIG. 1B illustrating an additional count in tablet entry screen configured for entry of a second surgical article type as compared to the count in tablet entry screen of FIG. 17.

FIG. 22 is a view of the display interface of FIG. 1B illustrating the count in summary page of FIG. 19 updated to reflect entry of the second surgical article type.

FIG. 23 is a view of the primary display screen of FIG. 1A illustrating the surgical procedure summary screen of FIG. 12, wherein the summary screen reflects the second surgical article type that has been entered into the procedure.

FIG. 24 is a view of the display interface of FIG. 1B illustrating a prompt to initiate a verbal count in process to double-check the results of the tablet count in process.

FIG. 25 is a view of the display interface of FIG. 1B illustrating a tablet count versus verbal count comparison screen configured for identifying any discrepancy between the results of the verbal count in process and the tablet count in process.

FIG. 26 is a view of the primary display screen of FIG. 1A illustrating the surgical procedure summary screen of FIG. 12. The summary screen reflects an identified discrepancy between count processes and provides details of the count.

FIG. 27 is a view of the display interface of FIG. 1B illustrating a discrepancy resolution screen. Details of an investigation into a cause of a discrepancy in the count may be annotated and the count may be corrected.

FIG. 28 is a view of the display interface of FIG. 1B illustrating a count out summary screen summarizing a state of a count out process and, in particular, provides a quantity of each surgical article type remaining to be counted out.

FIG. 29 is a view of the display interface of FIG. 1B illustrating a count out tablet entry screen enabling a user to remove surgical articles from the count with the tablet during a count out process.

FIG. 30 is a view of the display interface of FIG. 1B illustrating the dashboard view of FIG. 3. An option to end the present case or the present surgical procedure is currently selected.

FIG. 31 is a view of the display interface of FIG. 1B illustrating a case summary screen, illustrating count totals and conclusion of a successful count out process.

FIGS. 32A and 32B are each a portion of a flowchart, which together illustrate a method for implementing adjunct technology to facilitate a surgical procedure. A microphone may be used to record and interpret a confirming verbal count to summarize what is said during a count in process and using the confirming verbal count to verify a count entered by tablet.

FIG. 33 is a perspective view of a camera device being utilized to convert text on a physical object into machine-coded text and/or machine-coded values for use in conversion into an electronic count.

FIG. 34 illustrates a tabulation sheet which may be used to collect handwritten count notations for conversion to machine-coded text and/or machine-coded values.

FIG. 35 is a view of the display interface of FIG. 1B illustrating an electronic count created by handwritten text recognition and confirmation of the electronic count.

FIGS. 36A, 36B, and 36C illustrate a handwritten count in performed during a surgical procedure and representing a cumulative tally which may accrue over a period of time by the capturing and interpreting of a sequence of camera images of the handwritten count.

FIG. 37 is a flowchart illustrating a method for converting a handwritten count in into machine-coded text and machine-coded values for use in creating an electronic count based upon text recognition of the handwritten count.

FIG. 38 is a perspective view of a first example of an operating room including a first sterile field marked upon a floor of the operating room and a second sterile field defined by geofencing programming.

FIG. 39 is a perspective view of a second example of an operating room with an alternative sterile field defined by a distance from a center of an operating table.

FIG. 40 schematically illustrates an exemplary computerized device configured to recognize handwritten notations related to a count of surgical articles, wherein the computerized device is configured to operate a machine learning algorithm configured to adapt and iterative improve recognition of data.

FIG. 41 schematically illustrates the computerized device processing operational data after training of the device is complete.

### DETAILED DESCRIPTION

### I—System Overview

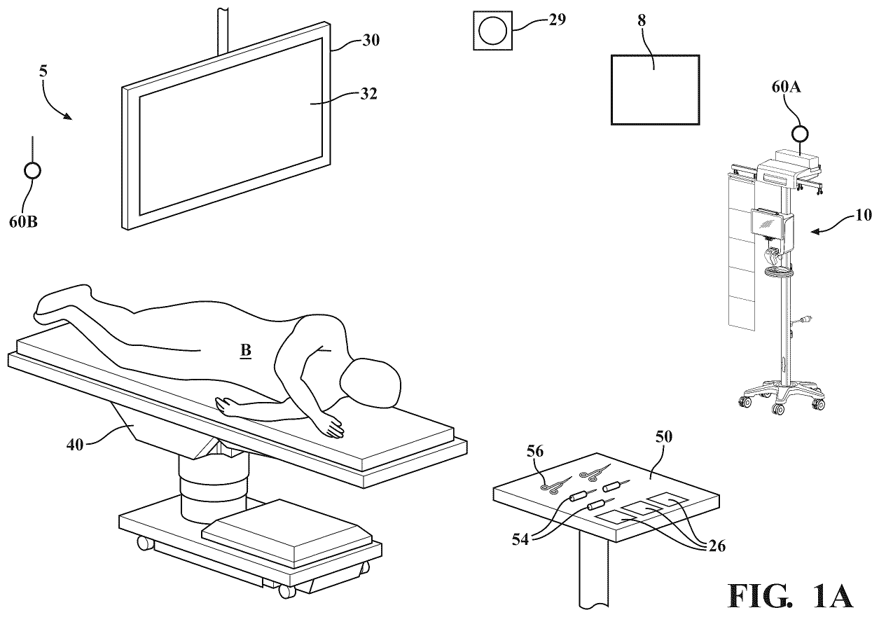

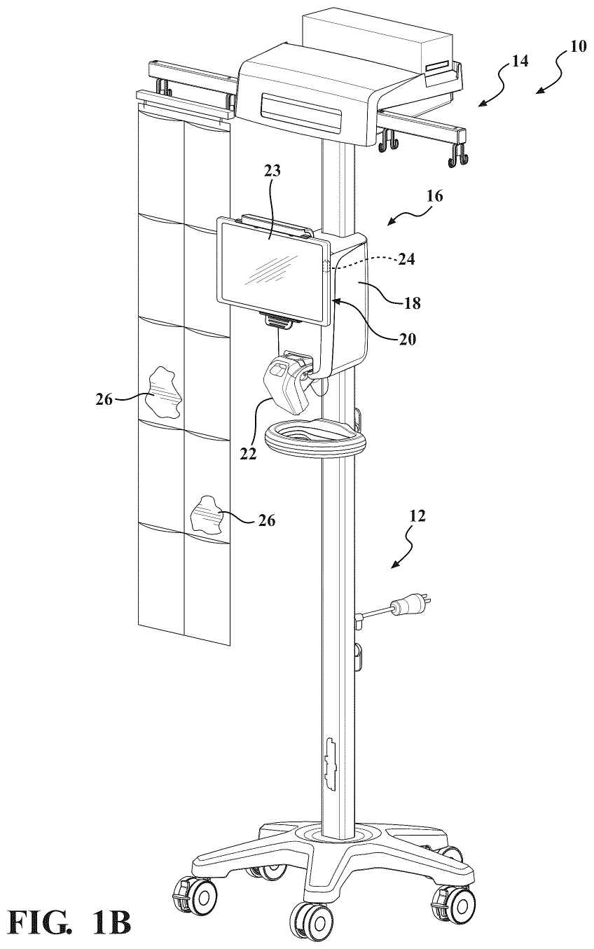

FIG. 1A depicts a surgical system 5 in a preparatory state for medical professionals to perform a surgical procedure upon a patient's body B. The surgical system 5 is able to monitor or track occurrence, location, movement, or other factors regarding objects and devices used to perform the medical procedure. The surgical system 5 may establish the location of an operating table 40 and register a location an object relative to the operating table 40. To these ends, and as is depicted schematically in FIG. 1A, the surgical system 5 may include one or more computerized devices 8 configured to operate programmed instructions. Each of the computerized devices may include a processor, random access memory (RAM), and durable memory configured to store programming and related data. In one embodiment, a computerized device 8 may include a server device located either locally or remotely and in electronic communication with the rest of the surgical system 5. The computerized device 8 may include or have access to information such as a digital catalog of surgical articles that may be utilized in a surgical procedure. The computerized device 8 may additionally include information, such a list of registered users; voice samples for those users; files including sign-in lists, time out lists, and check-out lists; and may include other information not specifically described but useful for the methods and processes described herein.

As is depicted schematically in FIG. 1A, the surgical system 5 includes a surgical sponge management system 10 including a computerized controller configured to aid in counting in and counting out of surgical sponges during the medical procedure. In the illustrated embodiment, the surgical sponge management system 10 may be disposed in communication with other components of the surgical system 5, such as via physical electrical connections (e.g., a tethered wire harness) and/or via one or more types of wireless communication (e.g., with a WiFi™ network, Bluetooth®, a radio network, and the like). The surgical sponge management system 10 and/or computerized components of the surgical system 5 may be realized as or with various arrangements of computers, processors, control units, and the like, and may comprise discrete components or may be integrated (e.g., sharing hardware, software, inputs, outputs, and the like). Other configurations are contemplated.

The surgical system 5 is further illustrated including a tabletop 50 including a plurality of surgical articles which may be counted in and counted out of the surgical procedure to be performed. The surgical accessories may include without limitation surgical sponges 26, a surgical drape, sharps 54, and surgical instruments 56 such as forceps and scalpels.

The surgical system 5 is configured to, among other things, track movement of various objects, such as sponges, sharps, and surgical instruments. The surgical system 5 may comprise other types of sensors and input devices, for example, including one or more camera devices configured to capture visual images and one or more microphones 60A, 60B configured to collect sounds and speech in an operating environment. In one embodiment, objects and devices may be equipped with tracker devices utilized in the art to provide location and orientation data based upon visual images captured of the tracker devices. A camera device 29 is illustrated configured for capturing images of objects and people within the OR.

The surgical system 5 may include a primary display 30 including a primary display screen 32 and configured to display relevant data and information collected and determined by the surgical system 5. In one example, the primary display 30 may include information about the patient, may include information about the medical professionals signed into the procedure, details regarding the surgical procedure being performed, checklist information for a particular stage of the procedure being performed, and information such as a list of surgical accessories counted in and surgical accessories counted out of the procedure. The primary display 30, in one embodiment, may be a display screen or a plurality of display screens displaying information which may be mounted to a wall, suspended from a ceiling, mounted upon a wheeled cart, or otherwise disposed within an operating room in which the procedure is taking place. In one embodiment, the primary display 30 may be described as a “main television” or “main TV” within the operating room.

In some embodiments, the surgical system 5 may include transmitters and sensors that are radio frequency (RF) based. Exemplary trackers installed upon surgical accessories may comprise RF emitters or transponders, which may be passive or may be actively energized. The RF transceiver transmits an RF tracking signal, and the RF emitters respond with RF signals such that tracked states are communicated to (or interpreted by) the surgical system 5. The RF signals may be of any suitable frequency. The RF transceiver may be positioned at any suitable location to track the objects using RF signals effectively. Furthermore, it will be appreciated that embodiments of RF-based navigation systems may have structural configurations that are different than the embodiments illustrated herein. In some embodiments, sensors of the surgical system 5 may additionally or alternatively be electromagnetically (EM) based.

In some embodiments, the surgical system 5 is capable of displaying a virtual representation of the relative positions and orientations of tracked objects to the surgeon or other users of the surgical system 5, such as with images and/or graphical representations of the anatomy of the patient's body B and the surgical accessories being tracked. The surgical system 5 may be useful to record a verbal sponge count, initiate a timer sequence, or request information on a surgical accessory. Other configurations are contemplated.

FIG. 1B shows a surgical sponge management system 10 including a stand 12, a dispenser assembly 14, and an electronics subsystem 16. The stand 12 supports the dispenser assembly 14 configured to store and ergonomically dispense sponge sorters and surgical draping. The electronics subsystem 16 includes a module base 18, a tablet device 20, the data reader 22. The tablet device 20 may be removably coupled to the module base 18. The tablet device 20 acts as a graphical user interface (GUI). The tablet may include a processor 24, or alternatively the tablet may provide wireless connectivity with remote resources, such as a hospital network or internet server, for remote processing. The tablet device 20 may include a camera and the microphone 60A of FIG. 1A. The module base 18 may include a cradle configured to be removably coupled with the data reader 22. The illustrated implementation shows the cradle being a recess sized to receive and support the data reader 22 such that the data reader 22 is configured to be used as either a handheld device or while supported by the cradle, and seamlessly transition between the configurations. The primary display 30 of FIG. 1A may be positioned elsewhere within the operating theatre. The illustrated surgical sponge management system 10 may be exemplary, and other similar embodiments may be described for counting other surgical articles which may be dispensed in quantity during a surgical procedure.

The data reader 22 may be an RFID scanner configured to detect RFID tags 120 associated with sponges 26. The RFID scanner includes the physical components and the operating software for generating interrogation signals and receiving responses to the interrogation signals. The physical components may include a signal-generating transmitter, and a signal receiver or transceiver, for example, as disclosed in commonly-owned International Publication No. WO2021/041795, published Mar. 4, 2021, and commonly-owned International Publication No. WO2021/097197, published May 20, 2021, each of which is hereby incorporated by reference in its entirety. Exemplary tags are disclosed in commonly-owned U.S. Pat. No. 8,181,860, published Oct. 2, 2008, and International Publication No. WO2017/112051, published Jun. 29, 2017, the disclosure of each being hereby incorporated by reference in its entirety. The surgical sponges 26 or sponge pads, as used herein, may be any absorbent article, including but not limited to laparotomy pads, gauzes, towels, chux, and the like. Further, it should be understood that, as an alternative to surgical sponges, aspects of the present disclosure may be modified to be used with non-absorbent surgical articles including implants, clips, staples, or surgical instruments. For example, common-owned United States Publication No. 2019/0000589, published Jan. 3, 2019, hereby incorporated by reference, discloses implementations in which a scannable element and a human-readable element are disclosed on forceps and suture needles.

The surgical procedure—and the procedural protocols corresponding thereto—may include different phases, for example, preprocedural check-in, sign-in, time-out, and sign-out. Within those phases, the procedural protocols may indicate one or more steps, such as confirmation of patient identity, confirmation of surgical site, item counts, and the like. The system 10 of the present disclosure may facilitate advancing through the procedural protocols and the phases of the surgical procedure in an efficient, intuitive, and error-preventing manner. Among other features to be described, the system 10 may require manually-performed steps to be confirmed with the adjunct technology, provide “lock outs” or triggers in the absence thereof, and provide visual indicators with successful or unsuccessful completion of various steps of the procedural protocols.

### II—Operation of the System

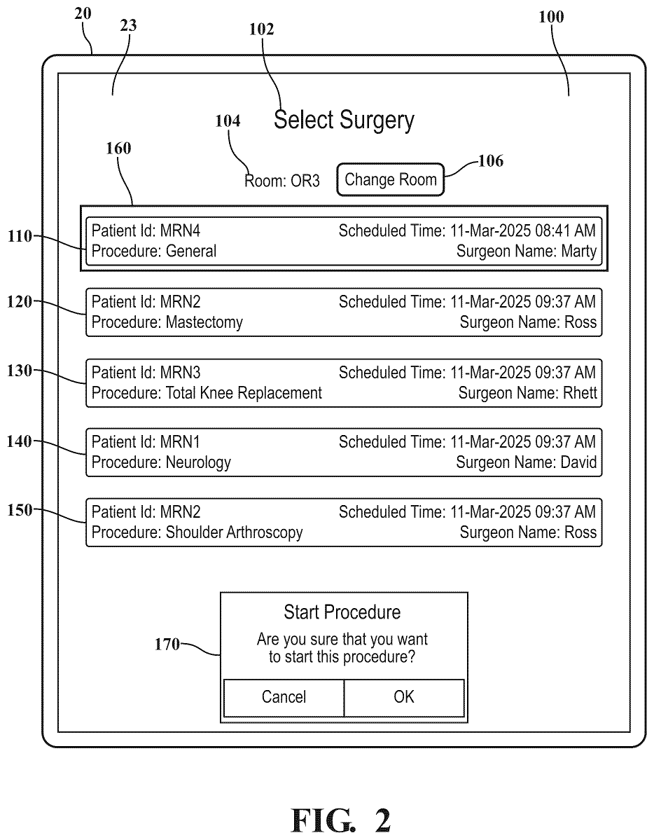

FIGS. 2-31 show exemplary progression through a plurality of display screens that may be provided to staff through the tablet device 20 and the primary display 30 over the course of a surgical procedure. FIG. 2 is a view of the display interface 23 of FIG. 1B, illustrating a plurality of selectable planned surgical procedures 110, 120, 130, 140, 150 that are scheduled for a particular operating room. The surgical system 5 of FIG. 1A may be utilized to plan and operate a plurality of surgical procedures, each for one of a plurality of patients. In the example of FIG. 2, a plurality of surgical procedures may be grouped by an operating room (OR) in which the procedures are set to take place. The tablet device 20 is illustrated including a display interface 23 may be embodied as a touch screen configured to display graphical images and simultaneously receive touch inputs to defined portions of the screen. A surgery procedure selection screen 100 is illustrated. A banner 102 defines the screen as currently permitting one to select a particular planned surgery procedure from a plurality of planned surgery procedures. Banner 104 defines the current OR in which the plurality of planned surgery procedures is set to place as OR3. Button 106 permits a user to change the display to describe procedures planned for a different OR. The plurality of planned surgical procedures is illustrated to include surgical procedures 110, 120, 130, 140, 150. Each of the surgical procedures 110, 120, 130, 140, 150 is associated with details including a patient identification (ID), a procedure description, a scheduled time, and a surgeon name. A selection box 160 indicates that surgical procedure 110 is currently selected. The figures herein utilize exemplary boxes such as the selection box 160 to illustrate a particular feature or graphical interface with which a user is illustrated as selecting. Such a box is used for purposes of illustration of user interaction with the display interface 23 of FIG. 1B or other human interface control surfaces. The actual display in such examples herein may or may not actually display a box around the feature or graphical interface being activated. The surgical system 5 is illustrated providing a confirmation box 170, requesting the user to confirm that the currently selected procedure 110 is desired to be started.

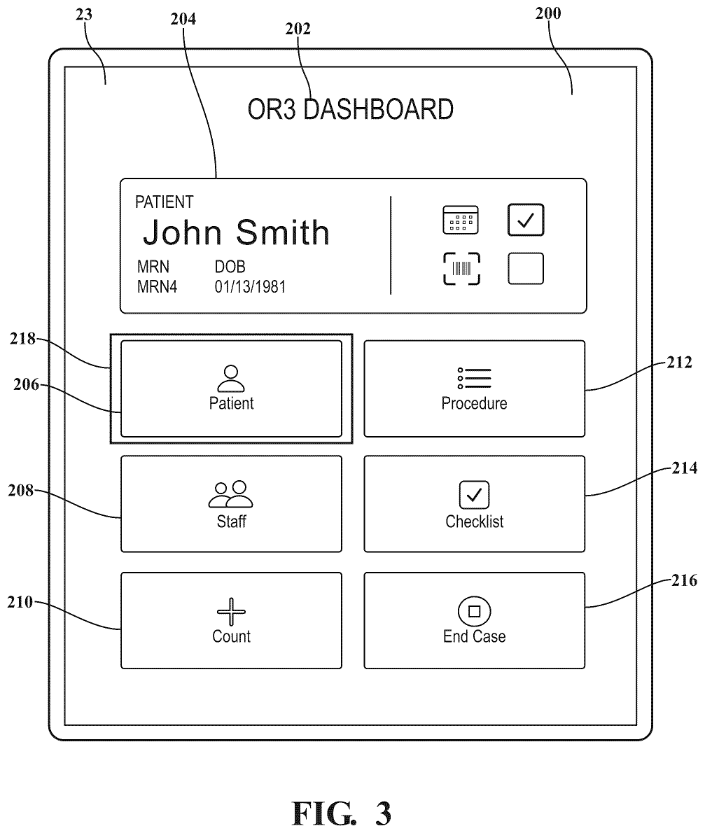

FIG. 3 is a view of the display interface 23 of FIG. 1B, illustrating a dashboard view 200, configured to enable one to manage the surgical system 5 throughout a surgical procedure, wherein an option to review details regarding the patient 206 is currently selected. The dashboard view 200 includes a banner 202 identifying the dashboard view and information 204 about the patient. A plurality of options are illustrated upon the dashboard view 200, including the option to review details regarding the patient 206, an option to review options and information regarding the procedure 212, an option to review options and information regarding the staff to perform the procedure 208, an option to review checklists for the surgical procedure 214, an option to enter a surgical article count 210 to manage a number of surgical articles counted in and counted out of the surgical procedure, and an option to end the case 216. A selection box 218 is illustrated showing selection of the option to review details regarding the patient 206.

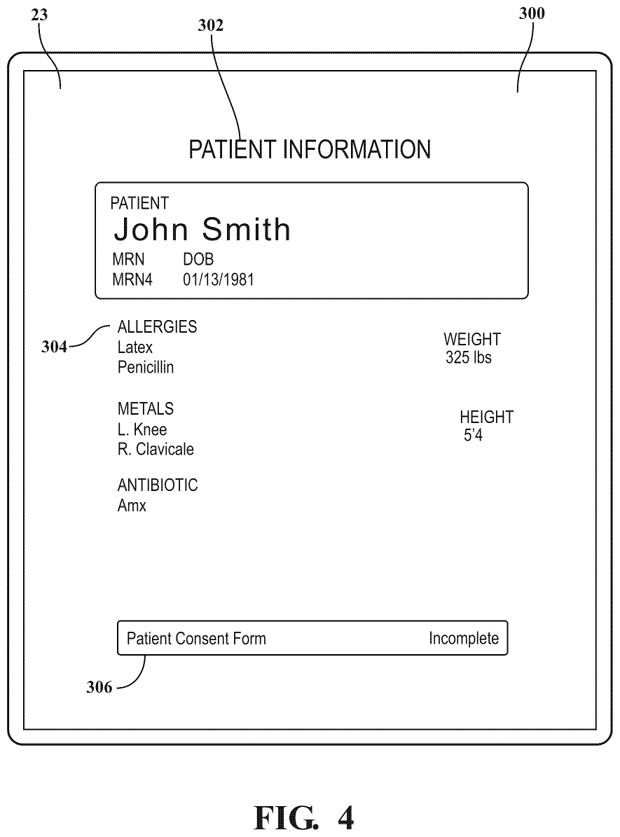

FIG. 4 is a view of the display interface 23 of FIG. 1B, illustrating a patient summary page 300 wherein details of the patient are listed and a status of a patient consent form is provided. A banner 302 identifying the page 300 as providing patient information is illustrated. Details about the patient 304 including exemplary information such as allergies, weight, portions of metal within the patient's body, height, and antibiotic administered to the patient are provided. Further, an indication 306 is provided indicating whether the patient has completed a patient consent form.

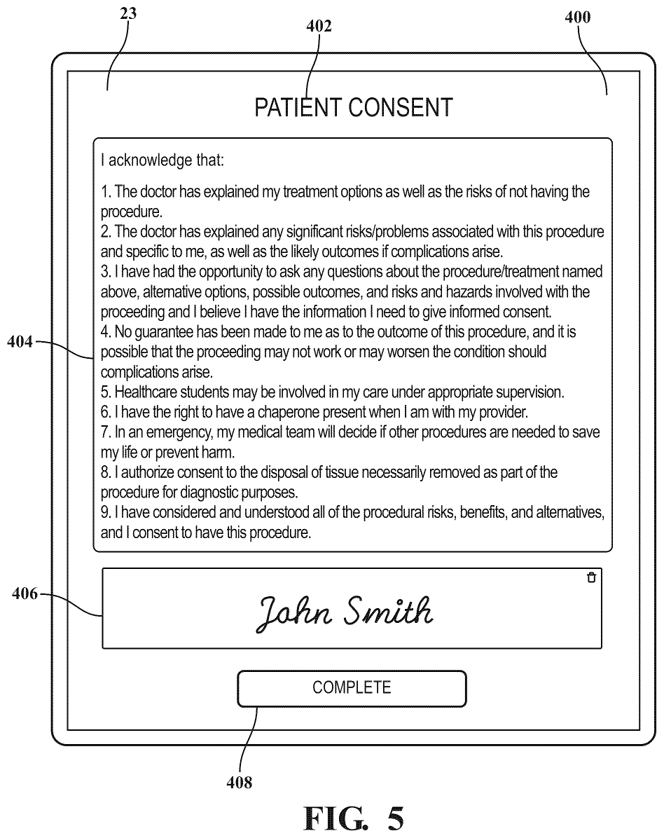

FIG. 5 is a view of the display interface 23 of FIG. 1B, illustrating an exemplary patient consent form 404 and a signature field 406 which may be presented to patient for touch screen signature. A patient consent page 400 includes a banner 402 describing the patient consent being sought. The patient consent form 404 is illustrated and provides terms of the agreement for consideration of the patient. The touch screen signature field 406 is provided, wherein the patient may make a signature mark, either with a finger or a provided stylus device. A confirmation is provided for the patient to tap as a complete button 408.

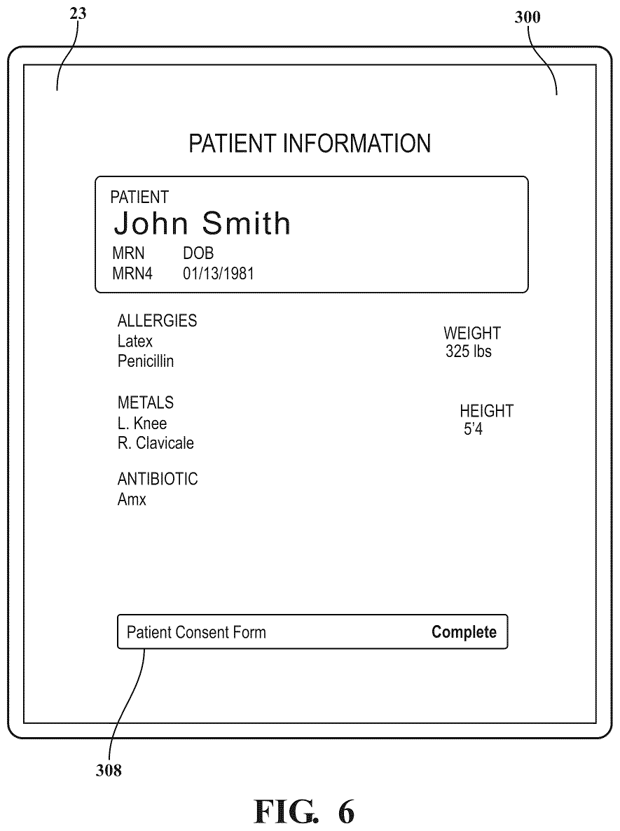

FIG. 6 is a view of the display interface 23 of FIG. 1B, illustrating the patient summary page 300 of FIG. 4, wherein the indication 306 is updated to reflect receipt of a signed patient consent form document.

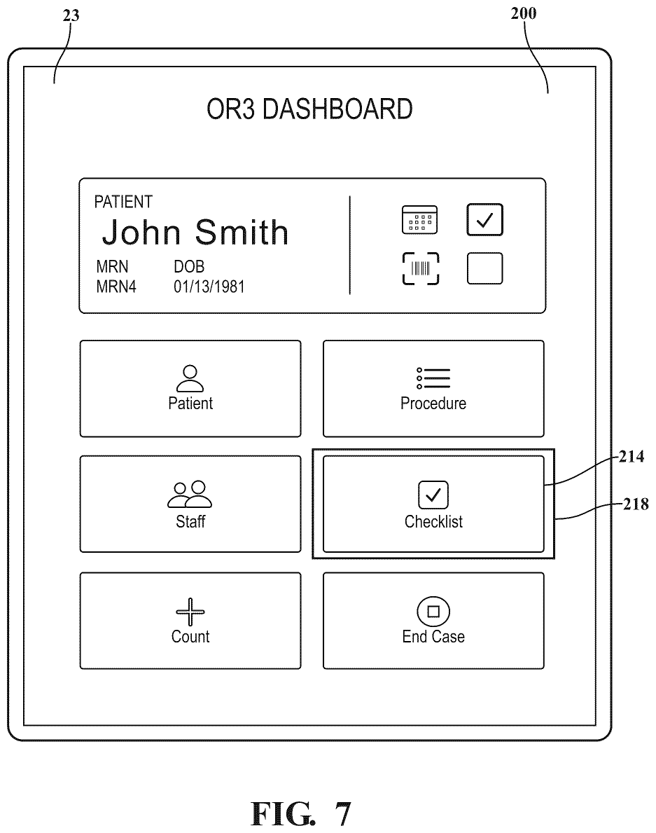

FIG. 7 is a view of the display interface 23 of FIG. 1B, illustrating the dashboard view 200 of FIG. 3, wherein an option to review checklists for the surgical procedure 214 is currently illustrated as being selected in accordance with location of the selection box 218.

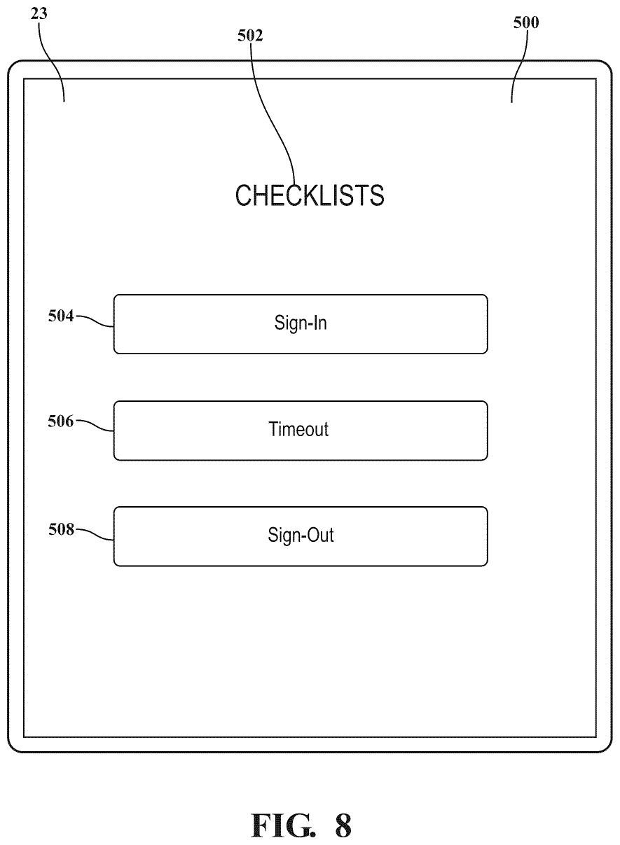

FIG. 8 is a view of the display interface 23 of FIG. 1B, illustrating a checklist summary page 500 providing an ability to select between various checklists that are available for the surgical procedure. A banner 502 is illustrated showing that the page summarizes available checklists. A sign-in checklist button 504, a timeout checklist button 506, and a sign-out checklist button 508 are illustrated and permit a user to navigate to each of the checklists.

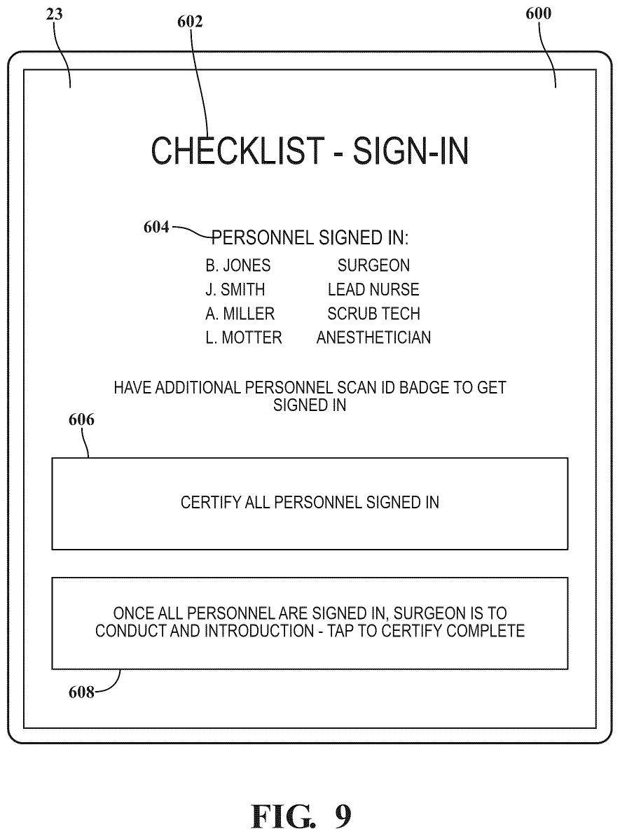

FIG. 9 is a view of the display interface 23 of FIG. 1B, illustrating an exemplary sign-in checklist. A sign-in checklist page 600 is illustrated including an identifying banner 602. A list of signed-in personnel 604 is provided describing which staff have signed in so far to the surgical procedure. A button 606 enables the staff to certify that all of the required personnel have signed-in. An optional button 608 is provided to certify that a surgeon for the surgical procedure has provided an introduction to clarify basic details of the procedure and/or the staff involved in the procedure.

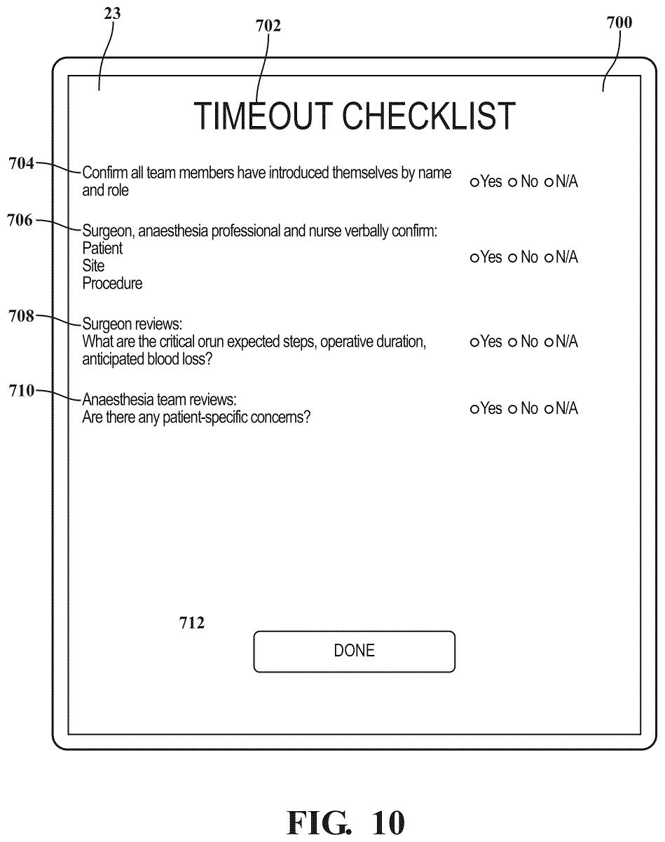

FIG. 10 is a view of the display interface 23 of FIG. 1B, illustrating an exemplary timeout checklist. A timeout checklist page 700 is illustrated including an identifying banner 702. In one embodiment, a timeout may be utilized to require that certain details of the planned surgical procedure are confirmed and that all of the staff are given an opportunity to receive the same basic information about the planned surgical procedure. A plurality of exemplary timeout questions 704, 706, 708, 710 are illustrated confirming that the staff have the correct information. Upon the timeout questions 704, 706, 708, 710 being satisfactorily answered, the timeout checklist may be completed through an illustrated button 712.

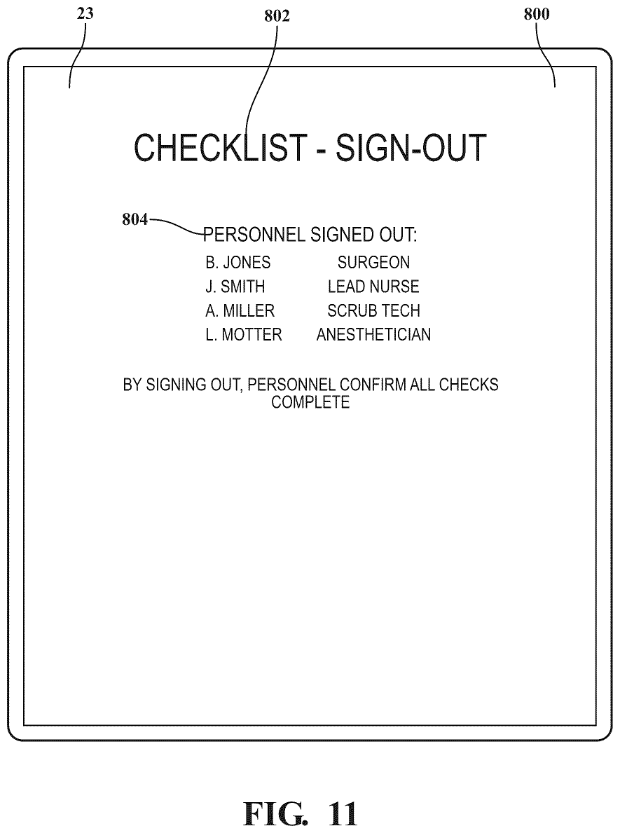

FIG. 11 is a view of the display interface 23 of FIG. 1B, illustrating an exemplary sign-out checklist. A sign-out checklist page 800 is illustrated including an identifying banner 802. A list of signed out staff 804 is illustrated confirming that each of the staff agree that the surgical procedure has been satisfactorily completed.

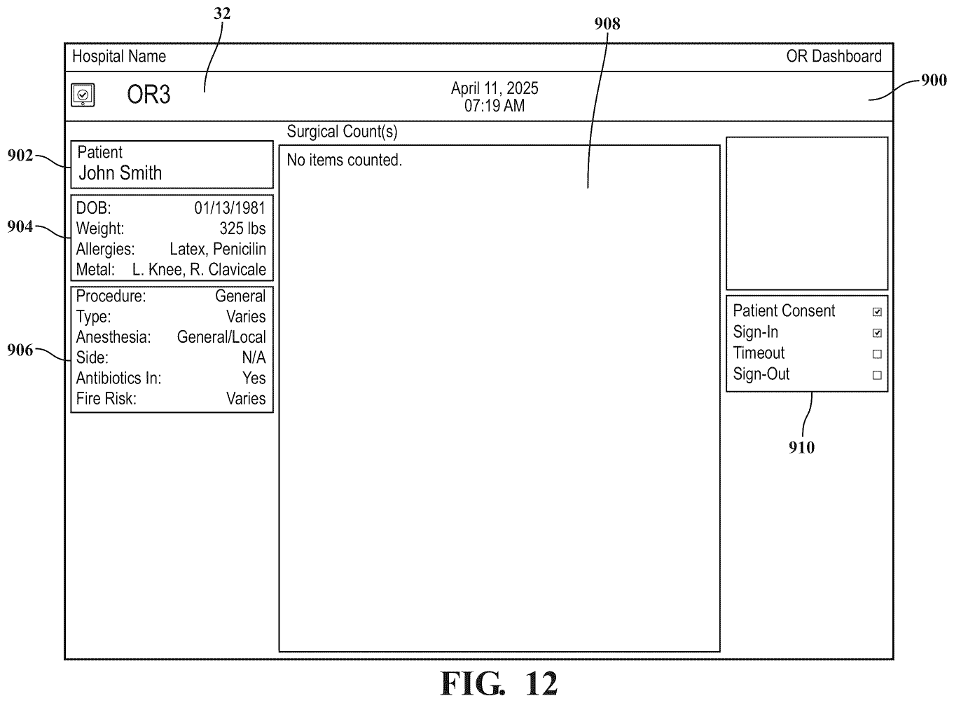

FIG. 12 is a view of the primary display screen 32 of FIG. 1A, illustrating a surgical procedure summary screen 900. The surgical procedure summary screen is configured to provide information to some or all of the staff in the OR regarding information relevant to the surgical procedure. A patient name 902, patient details 904, and surgical procedure details 906 are illustrated. A list of checklists for the procedure 910 include confirmation of whether the checklists have been complete is additionally illustrated. Further, a count board 908 is provided wherein details of surgical articles being counted in and counted out may be displayed.

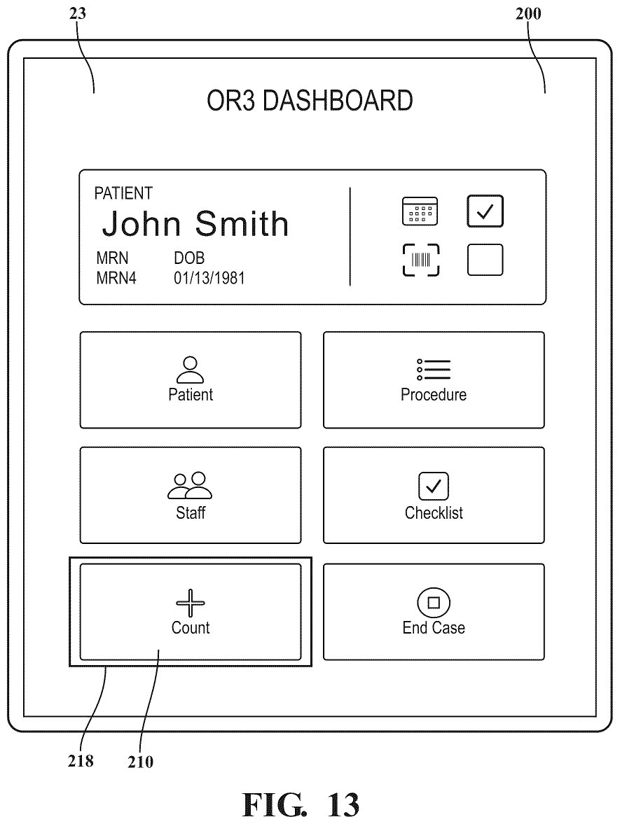

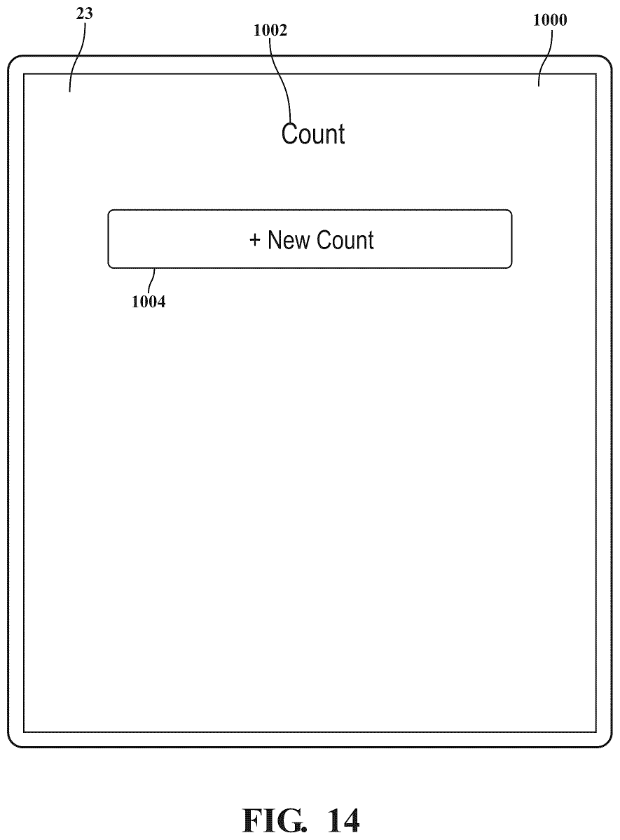

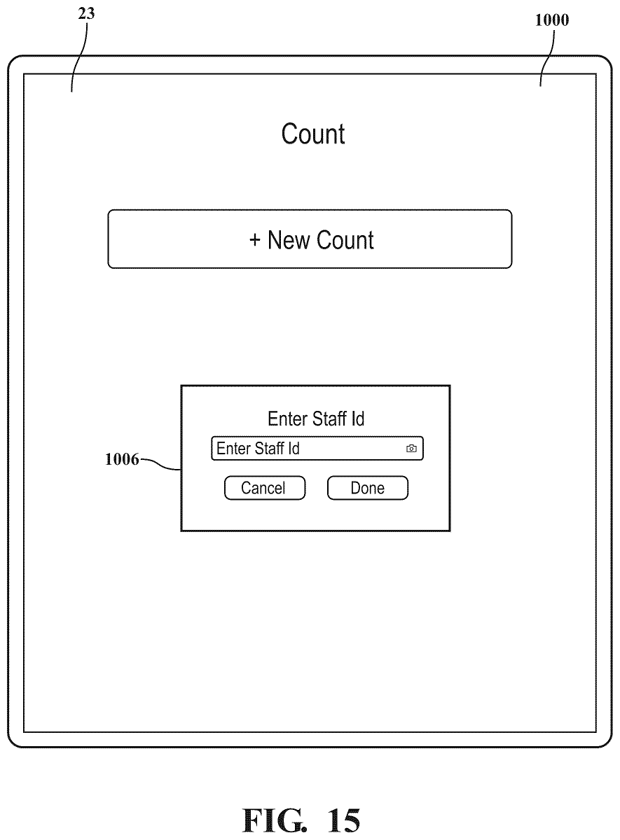

FIG. 13 is a view of the display interface 23 of FIG. 1B, illustrating the dashboard view of FIG. 3, wherein an option to enter a surgical article count 210 is indicated as selected by the selection box 218. FIG. 14 is a view of the display interface 23 of FIG. 1B, illustrating a new tablet count management screen 1000 including an identifying banner 1002. A button for initiating a new count 1004 is illustrated. FIG. 15 is a view of the display interface 23 of FIG. 1B, illustrating the new tablet count management screen 1000 of FIG. 14 including a staff ID registration window 1006 to correlate new count entries with a particular staff member. The staff member may be prompted to scan an ID card or to enter a personal staff ID number. By correlating new count entries with a particular staff member, any questions, discrepancies, or investigation later may be directed to the appropriate staff member as soon as possible and with as little confusion as possible.

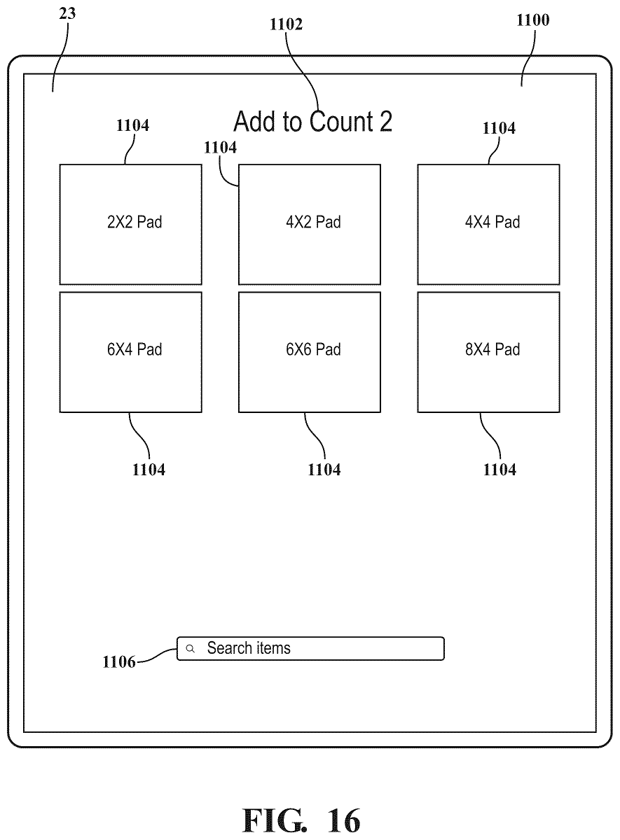

FIG. 16 is a view of the display interface 23 of FIG. 1B, illustrating a surgical article type selection screen 1100 for adding a new surgical article type to a count. An identifying banner 1102 is illustrated. A plurality of preconfigured or identified surgical article types 1104 are illustrated for selection. An entirety of available or selectable surgical article types may be provided. In another embodiment, a most-frequently-used or favorite list of surgical article types may be provided. Surgical article types may be referenced or added. The surgical article type selection screen 1100 is illustrated including an optional search field 1106. Selected surgical article types may be added from screen 1100 to a current count being tallied.

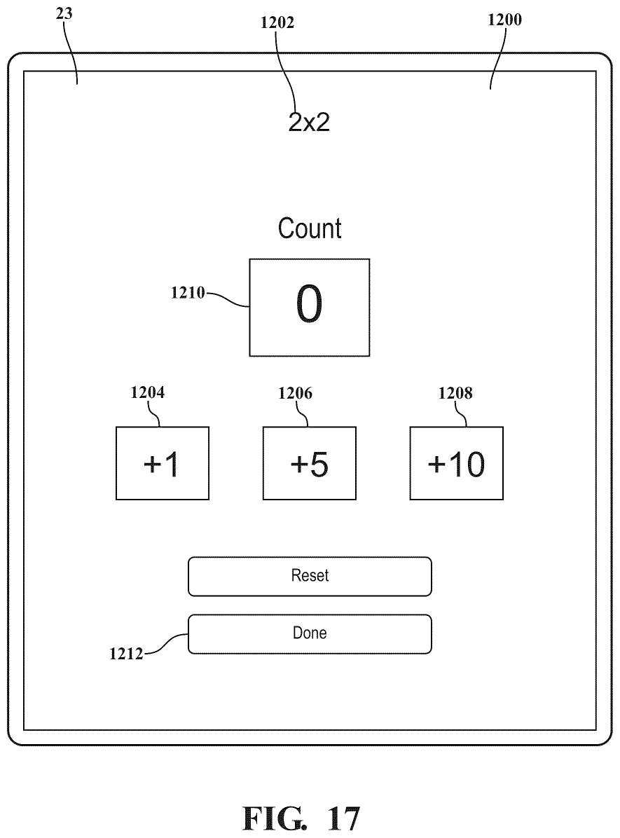

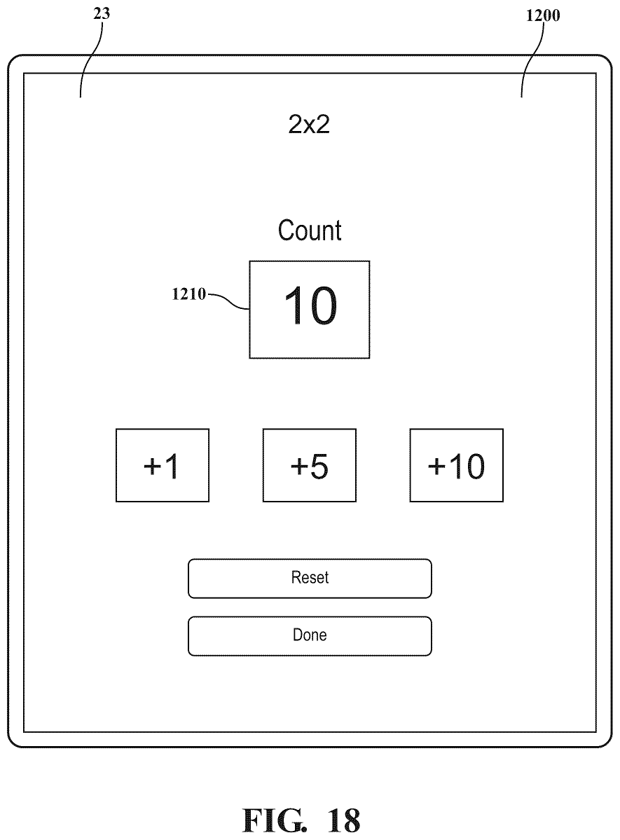

FIG. 17 is a view of the display interface 23 of FIG. 1B, illustrating a count in tablet entry screen 1200, enabling a user to add surgical articles to a count with the tablet device 20 of FIG. 1B during a count in process. An identifying banner 1202 is illustrated identifying a particular surgical article type currently being added to the count. In the example of FIG. 17, a two-inch by two-inch sponge pad is currently being added to the count. Quantity indicator 1210 illustrates that so far, none of the two-inch by two-inch pads have been added to the count yet. Buttons 1204, 1206, 1208 are provided adjacent to the quantity indicator 1210 and provide the user with a way to add to the present count a particular quantity, in this example, one piece, five pieces, and ten pieces, respectively, of the indicated surgical article. A reset button is provided to correct a count without entering the incorrect value, and a done button 1212 is provided to process the currently entered quantity value to the present count. FIG. 18 is a view of the display interface 23 of FIG. 1B, illustrating the count in tablet entry screen 1200 of FIG. 17, the present count.

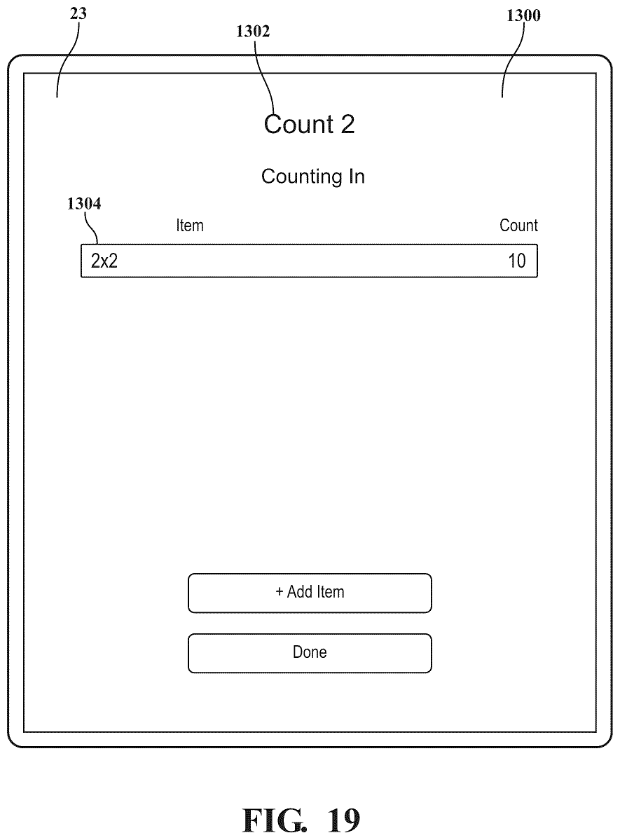

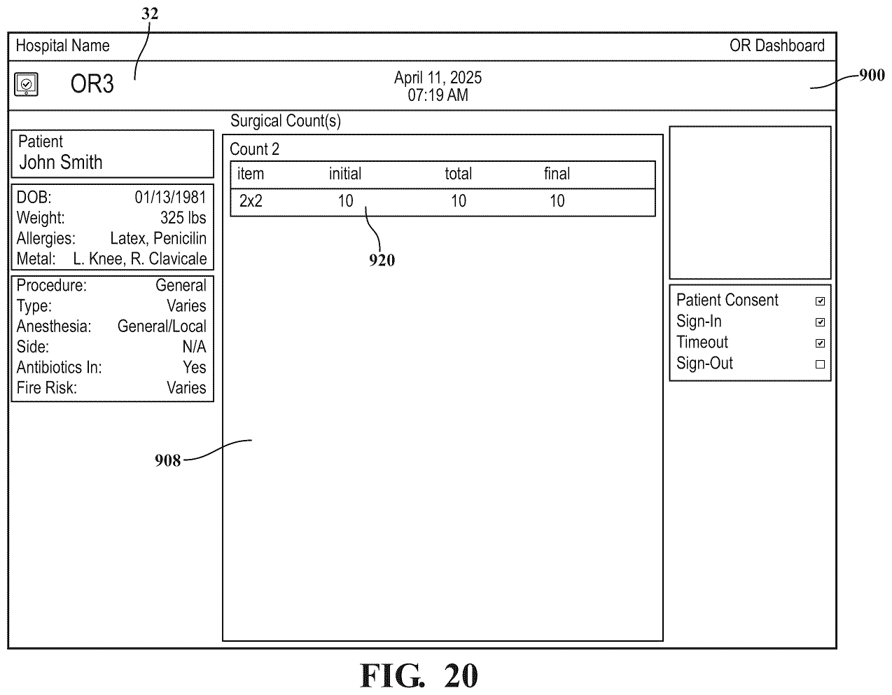

FIG. 19 is a view of the display interface 23 of FIG. 1B, illustrating a count in summary screen 1300 reflecting the exemplary ten two-inch by two-inch sponge pads that were entered into the count with the count in tablet entry screen 1200 of FIG. 18. An identifying banner 1302 is illustrated. Counted in entry 1304 provides a list of the currently counted in surgical articles including the described ten pads. FIG. 20 is a view of the primary display screen 32 of FIG. 1A, illustrating the surgical procedure summary screen 900 of FIG. 12, wherein the surgical procedure summary screen 900 reflects the exemplary ten two-inch by two-inch sponge pads that have been entered into the procedure. The count board 908 is illustrated updated with entry 920 which identifies the surgical article type and the quantity entered of that type.

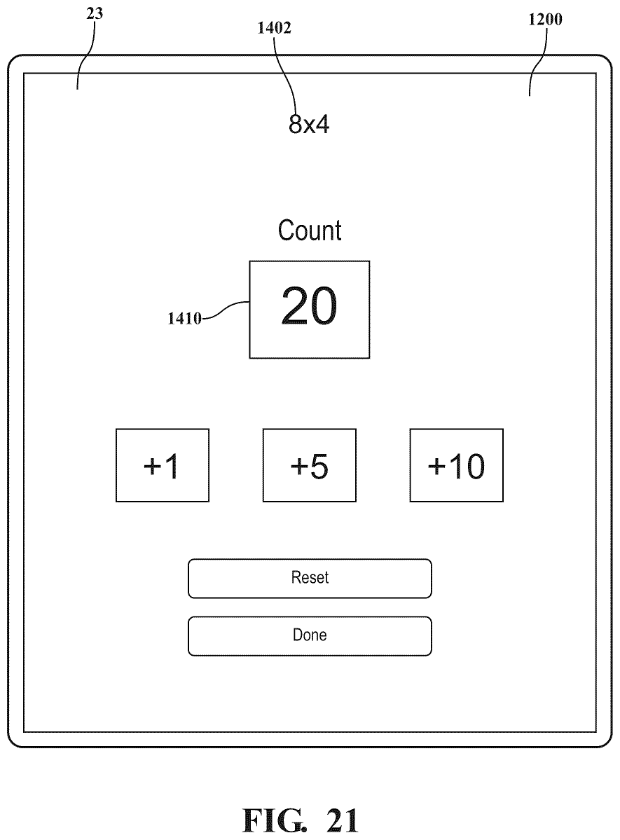

FIG. 21 is a view of the display interface 23 of FIG. 1B, illustrating an additional count in tablet entry screen 1200 configured for entry of a second surgical article type as compared to the count in tablet entry screen 1200 of FIG. 17. FIG. 21 includes an identifying banner 1402 describing that a surgical article type including eight-inch by four-inch sponge pads are currently being added. A quantity indicator 1410 indicates that twenty of the identified surgical articles are about to be added to the current count.

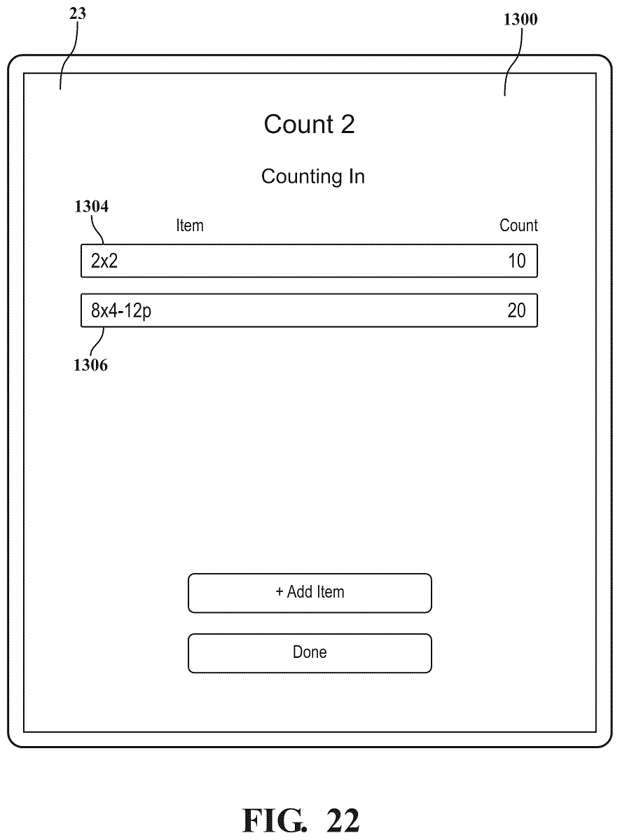

FIG. 22 is a view of the display interface 23 of FIG. 1B, illustrating the count in summary screen 1300 of FIG. 19 updated to reflect entry of the second surgical article type. Counted in entry 1304 provides a list of the currently counted in surgical articles including the previously described ten two-inch by two-inch pads. Additionally, the screen 1300 is updated to reflect a counted in entry 1306 describing the twenty eight-inch by four-inch pads that have been added. The count in summary screen 1300 may provide information related to a plurality of surgical article types and the quantities of each of the types that have been counted in. The surgical system 10 may manage a plurality of count ins at once (e.g., count 1, count 2, count 3) which may be defined by the user. In another embodiment, the counts may be pre-defined (count 1 describing sponges, count 2 describing sharps, etc.) Each count may be sorted separately, for example, with an ability for a user to quickly cycle thorough the active counts currently being tracked. The multiple counts may be listed together on a same display simultaneously. A user may be able to query across multiple counts, for example, instructing the system 10 to list all sponges currently counted in regardless of which count in the sponges are counted under.

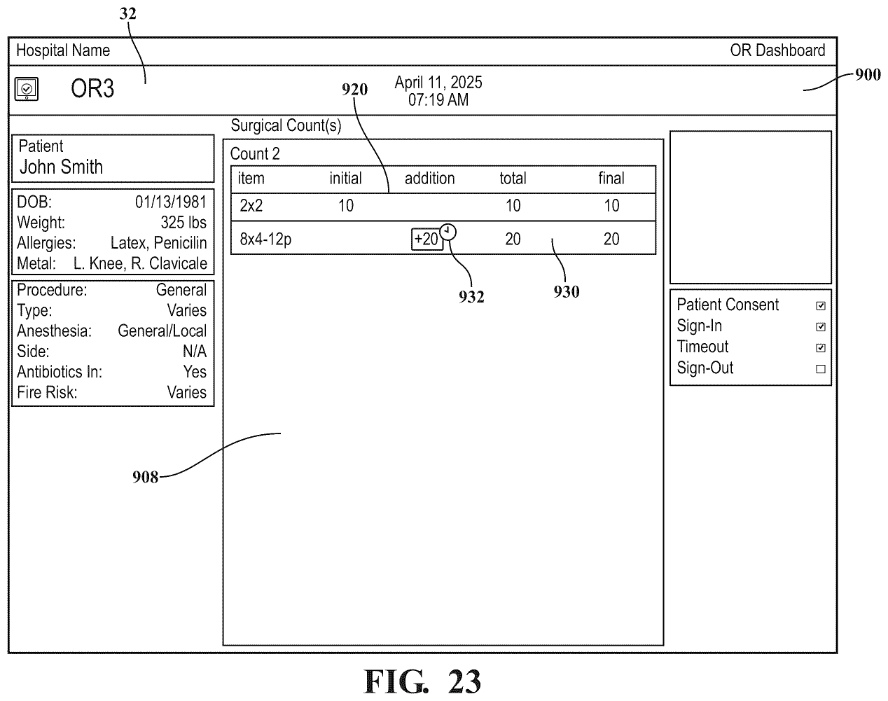

FIG. 23 is a view of the primary display screen 32 of FIG. 1A, illustrating the surgical procedure summary screen 900 of FIG. 12, wherein the summary screen 900 reflects the second surgical article type that has been entered into the procedure. The count board 908 is illustrated providing details of surgical articles that have been counted in to the surgical procedure so far. The count board 908 is illustrated displaying the details of entry 920, reflecting the two-inch by two-inch pads which were previously counted in, and the count board 908 is updated with entry 930 which identifies the surgical article type and the quantity entered of the eight-inch by four-inch which have recently been counted in. The entry 930 is further illustrated including a staff identity indicator 932, embodied as a dot next to the quantity counted in under the entry 930, wherein the dot is configured to identify a particular staff member with an example letter “J” displayed in the dot. Other initials, identifying numbers, thumbnail images of identification badge photo, or other representative image may be used with the staff identity indicator 932.

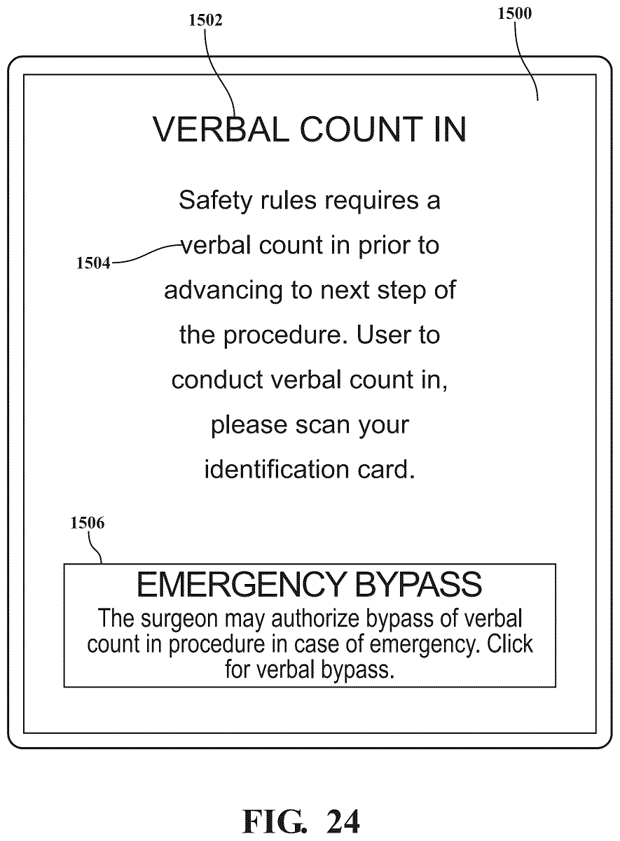

FIG. 24 is a view of the display interface 23 of FIG. 1B, illustrating a prompt screen 1500 to initiate a verbal count in process to double-check the results of the tablet count in process. The prompt screen 1500 is illustrating including a banner 1052 announcing a verbal count in process is active. The prompt screen 1500 further provides directions 1504 describing the requirement that a verbal count in prior to the procedure advancing to a next step. The prompt screen 1500 instructs that the user that is to conduct the verbal count in of surgical articles is to scan their identification card. In this way, the verbal count in may be performed at a selected stage of the surgical procedure, and a particular staff member is identified as having performed the verbal count in in case there are any questions or investigations conducted later. Surgical system 5 may thus implement a confirming verbal count, creating and using a real-time audio recording of the verbal count in, the audio recording summarizing what is said during a confirmation of the count in process, and analysis or speech conversion software enabling the contents of the audio recording to be verified on the tablet device 20 and the primary display 30 against the count in entered on the tablet device 20. An emergency bypass button 1506 is provided, enabling the surgeon to bypass the required verbal count, for example, if medical conditions for the patient warrant avoiding any time delay associated with the verbal count in process.

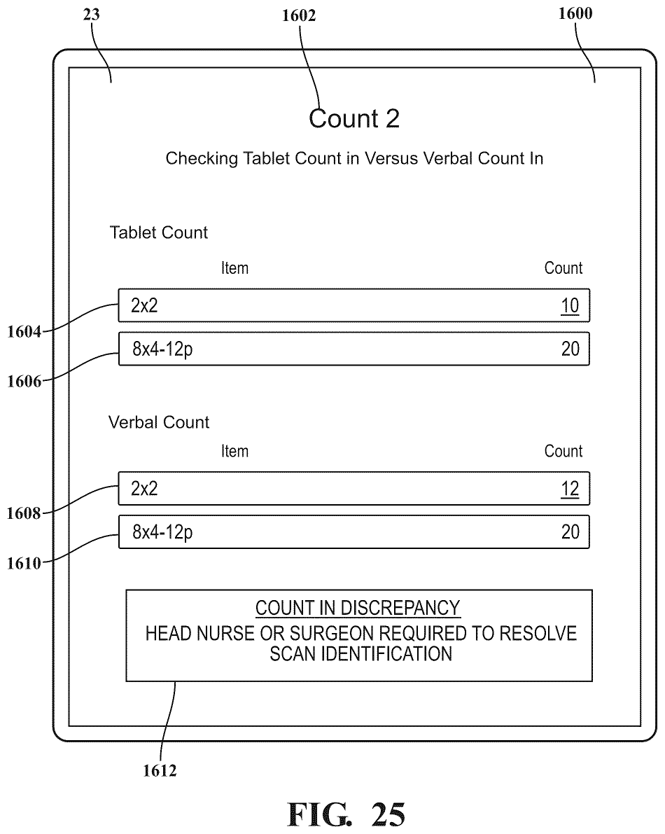

FIG. 25 is a view of the display interface 23 of FIG. 1B, illustrating a tablet count versus verbal count comparison screen 1600 configured for identifying any discrepancy between the results of the verbal count in process and the tablet count in process. The comparison screen 1600 is illustrated including a banner 1602 identifying a comparison being presented upon the comparison screen 1600. The comparison screen 1600 further illustrates tablet count in entries 1604 describing exemplary two-inch by two-inch pads that have been counted in by tablet and 1606 describing exemplary eight-inch by four-inch pads that have been counted in by tablet. The comparison screen 1600 further illustrates verbal count in entries 1608 describing exemplary two-inch by two-inch pads that have been counted in through a verbal count in process and 1610 describing exemplary eight-inch by four-inch pads that have been counted in by the verbal count in process. The system 5 may compare the quantity of each surgical article type counted in by each of the tablet count in process and the verbal count in process and identify any discrepancy between the quantities. In the example provided in FIG. 25, a discrepancy is identified in the quantity of two-inch by two-inch pads counted in by the tablet count in process, including ten pads, as compared to the quantity of two-inch by two-inch pads counted in by the verbal count in process, including twelve pads. The quantities “10” and “12” are illustrated highlighted, and a prompt 1612 is provided textually identifying a count in discrepancy and prompting appropriate personnel to being a process to resolve the discrepancy.

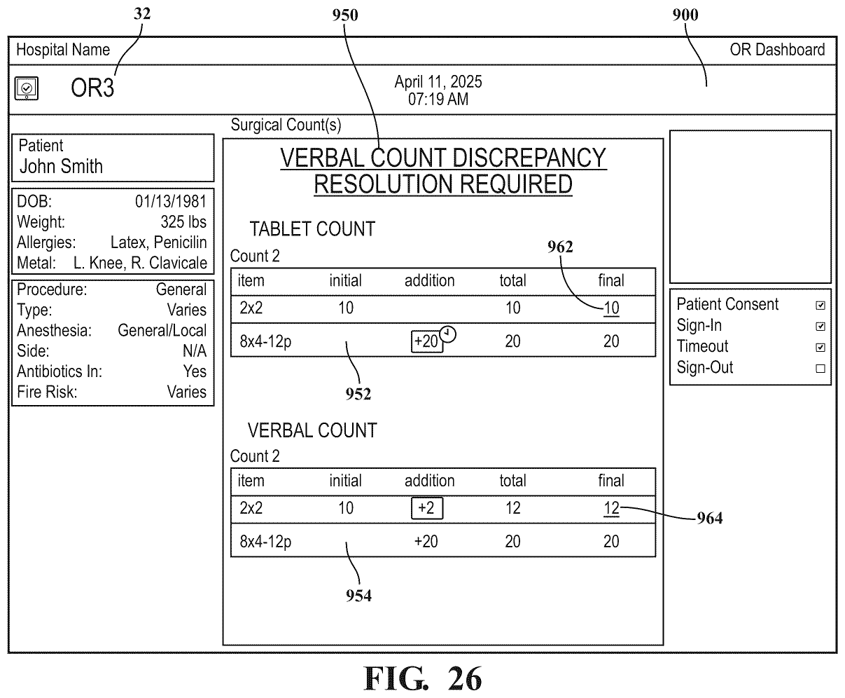

FIG. 26 is a view of the primary display screen 32 of FIG. 1A, illustrating the surgical procedure summary screen 900 of FIG. 12, wherein the summary screen 900 reflects an identified discrepancy between count processes and provides details of the count. The summary screen 900 is illustrated including a banner 950 describing an identified discrepancy. The summary screen 900 further illustrates the results of comparison data illustrated in FIG. 25. A tablet count in summary 952 is illustrated including a highlighted quantity 962, and a verbal count in summary 954 is illustrated including a highlighted quantity 964.

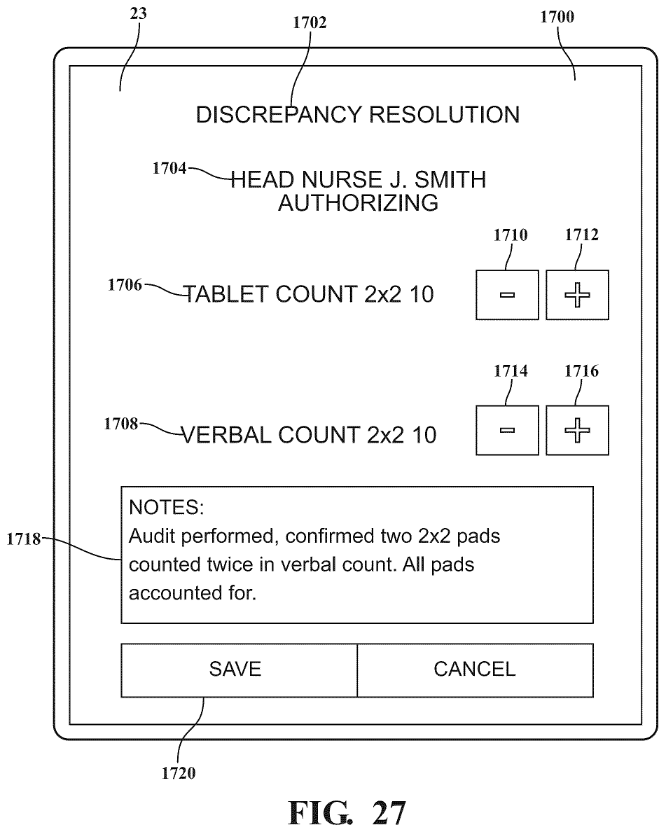

FIG. 27 is a view of the display interface 23 of FIG. 1B, illustrating a discrepancy resolution screen 1700, wherein details of an investigation into a cause of a discrepancy in the count may be annotated and the count may be corrected. The discrepancy resolution screen 1700 is illustrated including an identifying banner 1702 describing the discrepancy resolution process being undertaken. Text 1704 identifies a particular staff member that has taken responsibility for the discrepancy resolution process, in the example of FIG. 27, a head nurse. A tablet count in entry 1706 describing a surgical article type and a quantity is illustrated, with adjacent quantity adjustment buttons 1710, 1712 being provided. A verbal count in entry 1708 describing the surgical article type and a quantity is illustrated, with adjacent quantity adjustment buttons 1714. Personnel performing a discrepancy investigation into a cause and resolution for the identified discrepancy may correct the numerical values upon resolution screen 1700. Further, a field 1718 providing a space for text entry may be captioned with a title, such as “NOTES:” or “DOCUMENTED SOURCE OF DISCREPANCY:”, prompting the personnel to provide an explanation for why the discrepancy occurred and how it was resolved. Once the discrepancy has been resolved and the numbers have been corrected, the user may touch a save button 1720 to close the discrepancy resolution screen 1700.

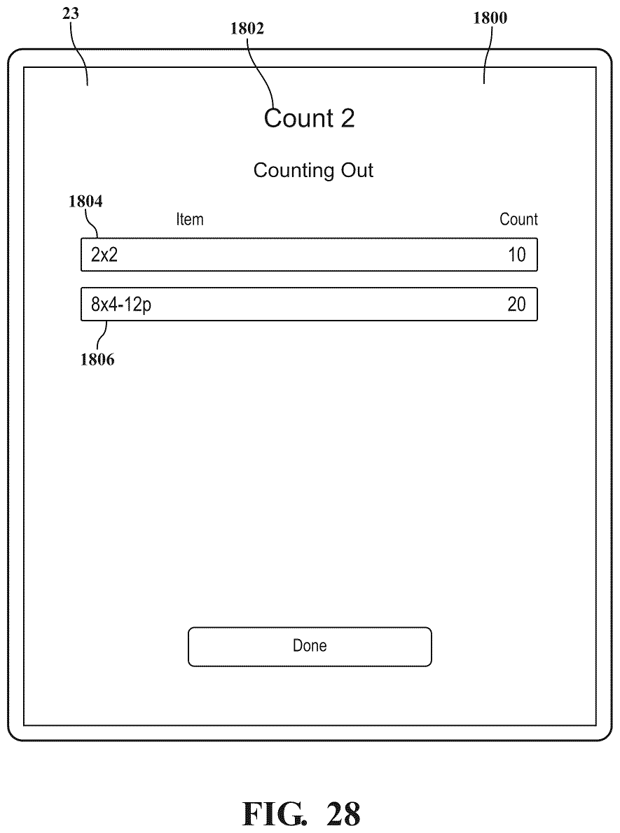

FIG. 28 is a view of the display interface 23 of FIG. 1B, illustrating a count out summary screen 1800, summarizing a state of a count out process and, in particular, provides a quantity of each surgical article type remaining to be counted out. The count out summary screen 1800 is illustrated including an identifying banner 1802, describing details such as identifying a count number and providing information that a counting out process has been initiated. A first entry 1804 and a second entry 1806 are provided, illustrating surgical article types associated with a particular count, in this case, count 2, that were counted in and providing a total quantity of each of the surgical article types that were counted in, respectively. Through the count out summary screen, the user may identify a total number of surgical articles that are to be counted out relative to the particular count.

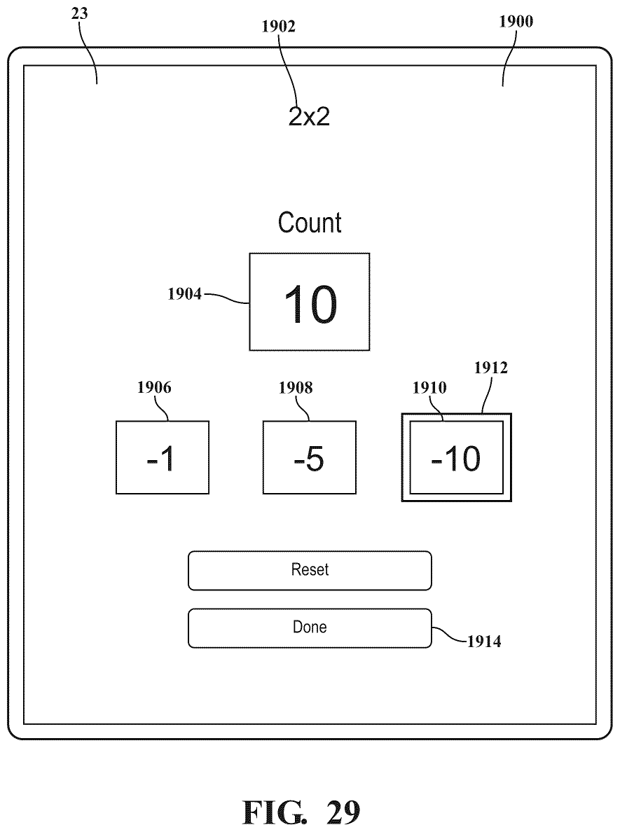

FIG. 29 is a view of the display interface 23 of FIG. 1B, illustrating a count out tablet entry screen 1900, enabling a user to remove surgical articles from the count with the tablet during a count out process. The count out tablet entry screen 1900 is illustrated including an identifying banner 1902 which may identify a surgical article type being counted out. A quantity indicator 1904 provides the user with a total number of the particular surgical article type that is still counted in to the surgical procedure. The user may utilize count out adjustment buttons 1906, 1908, 1910 to selectively document surgical articles as they are counted out of the surgical procedure. The count out adjustment button 1910 is highlighted with a selection box 1912. Upon activation of the count out adjustment button 1910, the user may transition the exemplary quantity indicator 1904 from ten to zero. Once the user has completed the count out process or intends to leave the count out tablet entry screen 1900 (possibly to come back later to complete the count out if the quantity indicator 1904 is not yet at zero), the user may touch a done button 1914.

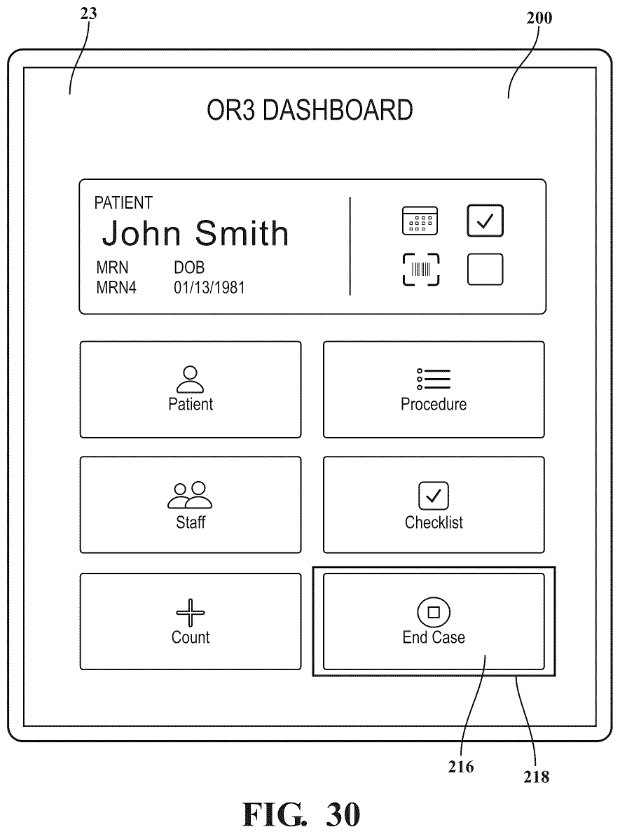

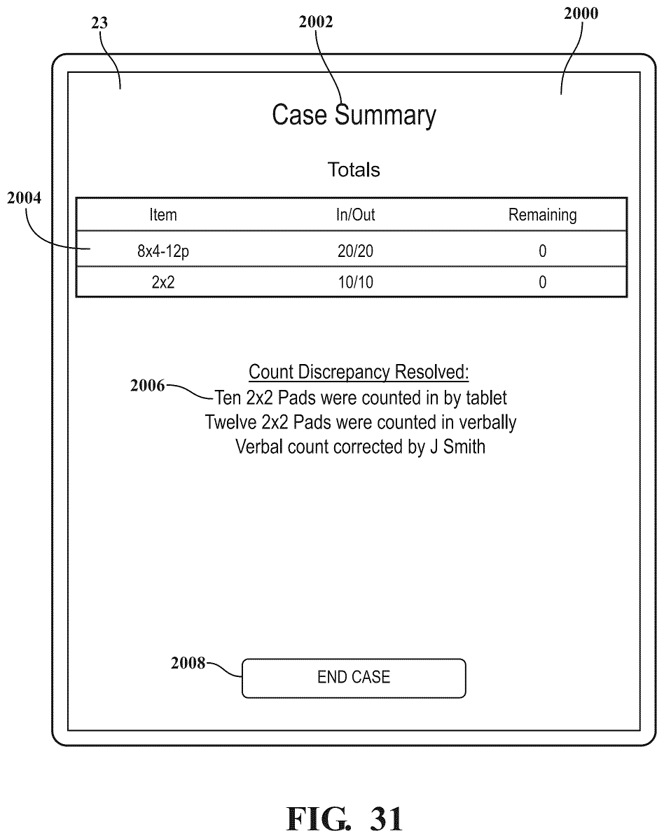

FIG. 30 is a view of the display interface 23 of FIG. 1B, illustrating the dashboard view 200 of FIG. 3, wherein an option 216 to end the present case or the present surgical procedure is currently selected. A selection box 218 is illustrated around the option 216, where a user may touch to indicate that the case is at an end. FIG. 31 is a view of the display interface 23 of FIG. 1B, illustrating a case summary screen 2000, illustrating count totals and conclusion of a successful count out process. The case summary screen 2000 is illustrated including an identifying banner 2002. A count summary 2004 is provided, describing a total number of surgical articles that were counted in and counted out during the surgical procedure. An optional display output 2006 may be provided to summarize any discrepancy that was resolved during the surgical procedure. An end case button 2008 is provided enabling the user to close the case. As a part of closing the case, results of the operation of system 5 may be saved for later review, investigation and for training purposes. In some embodiments, information such as audio recordings which may include private or confidential information may be encrypted for proper safeguarding of the patient's rights.

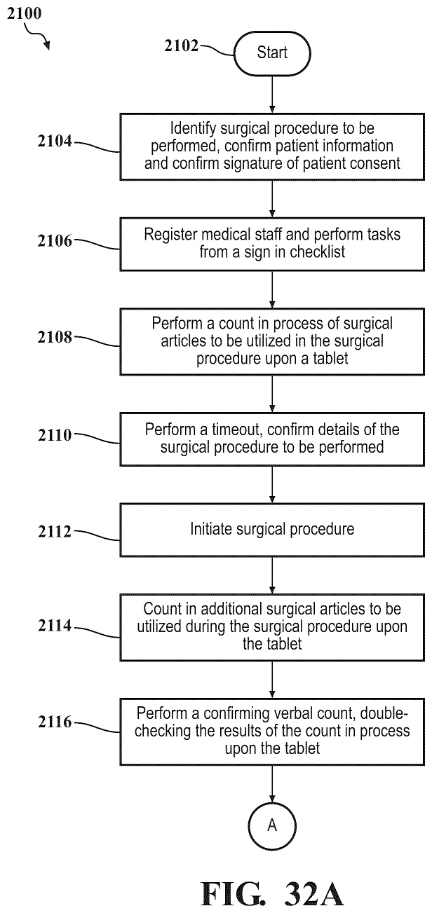

FIGS. 32A and 32B are each a portion of a flowchart, which together illustrate a method 2100 for implementing adjunct technology to facilitate a surgical procedure, including using microphone to record and interpret a confirming verbal count to summarize what is said during a count in process and using the confirming verbal count to verify a count entered by tablet. The method 2100 is provided capable of being operated with or upon system 5 of FIG. 1A, although it is intended that the method 2100 may be operated on other systems with other physical features and components. The method 2100 starts at step 2102. At step 2104, a surgical procedure to be performed is identified, patient information is confirmed, and a patient signature upon a patient consent form is confirmed. At step 2106, medical staff that will participate in the surgical procedure are registered, for example, by scanning in identification badges, and tasks provided on a sign-in checklist are performed. At step 2108, for example, as part of a pre-surgical process, a count in process of surgical articles to be utilized in the surgical procedure is performed on a tablet device. At step 2110, a time out is performed, for example, utilizing a time out checklist, wherein details of the surgical procedure to be performed are confirmed. At step 2112, the surgical procedure is initiated. At step 2114, additional surgical articles, in addition to the articles that were counted in at the step 2108, are counted in using the tablet device. At step 2116, a confirming verbal count is performed, double checking the results of the count in process upon the tablet. At step 2118, a determination is made whether the confirming verbal count matches the results of the count in process upon the tablet. If the results of the two processes match, the method 2100 advances to step 2120, wherein the surgical team may be provided with a report that the count in results match and that no discrepancy exists, and the method 2100 advances to step 2126. If the results of the two processes do not match, the method 2100 advances to step 2122, wherein the surgical team may be provided with a report that a discrepancy exists between the tablet count and the verbal count. At step 2124, a discrepancy resolution process is initiated, wherein count in totals are corrected based upon an investigation by the surgical team. At step 2126, the surgical procedure may be completed, during which the team may be prompted to conduct a body cavity search to confirm that surgical articles have been removed from the body cavity and a count out process may be performed. At step 2128, the system may confirm that the body cavity search was completed. At step 2130, the system may confirm that the count out process has been completed. At step 2132, a sign out checklist may be utilized, wherein tasks from the checklist may be performed and completed. At step 2134, the method 2100 ends. The method 2100 is provided as an example of how one may implement adjunct technology to facilitate a surgical procedure, including using microphone to record and interpret a confirming verbal count to summarize what is said during a count in process and using the confirming verbal count to verify a count entered by tablet. A number of additional and/or alternative method steps are envisioned, and the disclosure is not intended to be limited to the examples provided herein.

### III—Converting Hand-Written Count Information Using Optical Character Recognition Techniques

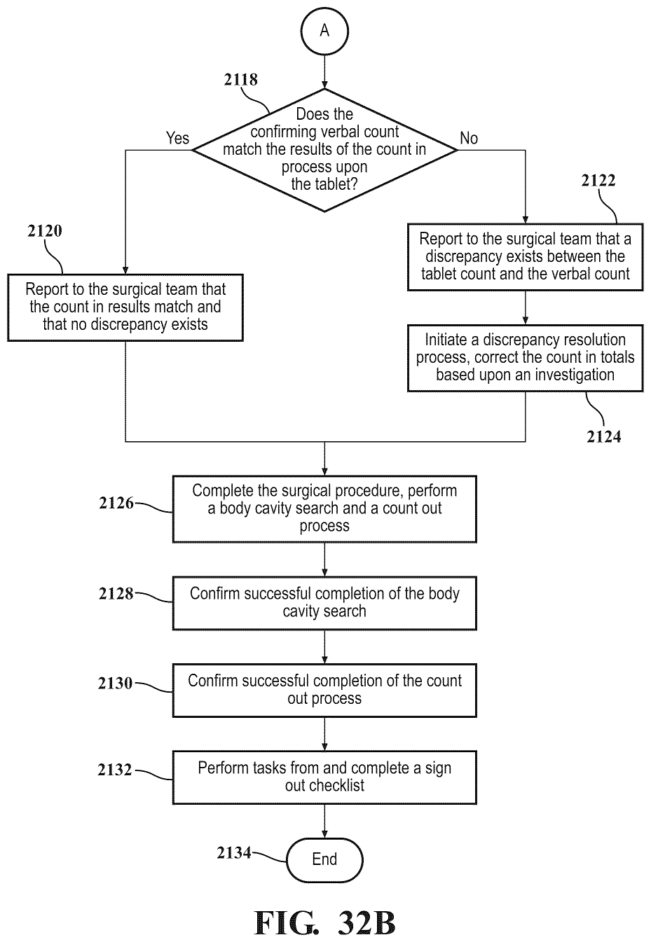

As described herein, a count in process may include utilizing a tablet to enter a count and a confirming verbal count may be utilized to double-check the count entered upon the tablet. Other counting processes may additionally and/or alternatively be utilized. In some embodiments, surgical teams may utilize handwritten notes to perform a count in process. Some handwritten notes may be recorded on a pad of paper or a printed form with fields provided for the user to write surgical article types and corresponding quantities. Some handwritten notes may be recorded on a whiteboard or dry-erase board. FIG. 33 is a perspective view of a camera device 2230 being utilized to convert text on a physical object into machine-coded text and/or machine-coded values for use in conversion into an electronic count. The camera device 2230 is illustrated including a field of view 2232 describing an area or region of which the camera device 2232 may capture or generate images of. Within the field of view 2232, a writing surface 2210 is illustrated. The writing surface 2210 may include any surface upon which a user may write information related to a count. In one embodiment, the writing surface may be a piece of paper. In another embodiment, the writing surface may be a whiteboard or dry-erase board. The camera device 2232 may be utilized in combination with the system 5 of FIG. 1A and may be additive to or may be the camera device 29 of FIG. 1A.

In the embodiment of FIG. 33, the writing surface 2210 is divided into sections 2212, 2214, 2216, 2218 or regions. Such sections or regions may be useful to enable one to annotate both surgical article types being counted and a corresponding quantity of the surgical article being counted in or being counted out. Such a physical or visual division of the writing surface 2210 may be useful to aid both the user and the system 5 to create and recognize, respectively, the count accurately. However, the writing surface 2210 and the notes included on the writing surface 2210 may vary, and the disclosure is not intended to be limited to the examples provided herein.

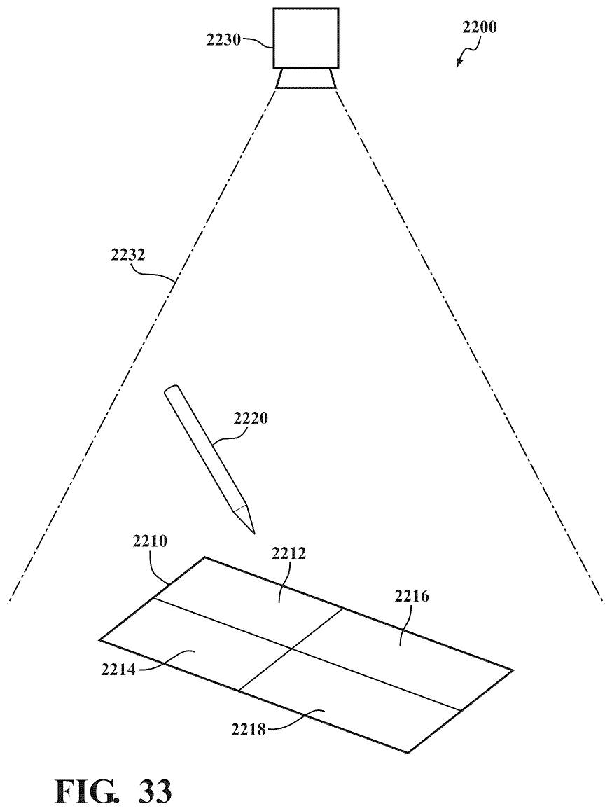

FIG. 34 illustrates the writing surface 2210 of FIG. 33 embodied as a tabulation sheet which may be used to collect handwritten count notations for conversion to machine-coded text and/or machine-coded values. The writing surface 2210 is illustrated including the sections 2212, 2214, 2216, 2218. In one embodiment, the writing surface 2210 may include a marker 2240 which may include a quick response (QR) code configured for storing and visually conveying data to a computer. Such a marker 2240 may enable the system 5 to access parameters of the particular writing surface 2210, for example, to preload dimensions of the writing surface 2210 to improve performance of software interpreting markings upon the writing surface 2210 and/or preload any pre-printed words or symbols upon the writing surface 2210. For example, a writing surface 2210 may be pre-printed with commonly used surgical articles, and markings upon the writing surface 2210 near to or proximate to the pre-printed annotations may be easily associated with the respective pre-printed surgical articles upon the writing surface 2210. An image of the marker 2240 and pre-printed graphics thereupon may additionally or alternatively facilitate the camera device 2230 and/or associate software in determining an orientation and distance of the writing surface 2210 from the camera device 2230, thereby improving recognition of handwritten noted upon the writing surface 2210.

FIG. 34 illustrates exemplary pre-printed column labels 2241, 2242, describing to a user that that a first column is to include surgical article types and that a second column is to include a quantity to count in, respectively. The example of FIG. 34 includes a pre-printed surgical article type, a “2×2 Gauze Pad” label 2243 printed in the section 2212. The section 2214 is illustrated including a handwritten notation 2244 describing “8×4”, which the camera device 2230 and associated software may determine to indicate an intent to count eight-inch by four-inch gauze pads. The section 2216 is illustrated including handwritten tick marks 2245 commonly used to count items or instances of occurrence. The second 2218 is illustrated including a handwritten annotation 2246 including a plurality of numbers with plus signs between the numbers, indicating an intention that the user intends the values of the numbers to be added together to create a total number of the respective surgical article being counted. The counting annotations of FIG. 34 are exemplary, and a variety of different counting annotations are envisioned and may be utilized.

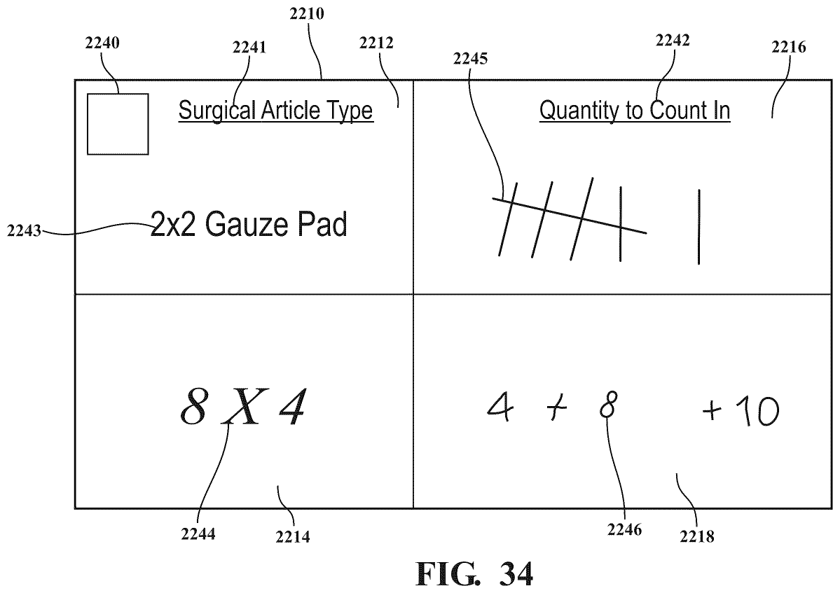

FIG. 35 is a view of the display interface 23 of FIG. 1B, illustrating an electronic count display 2300 created by handwritten text recognition and confirmation of the electronic count. The electronic count display 2300 is illustrated including an identifying banner 2302. A text portion 2304 identifies for the user that the system has captured images of handwritten notations. A tabular output 2306 identifies an example of a plurality of surgical article types and quantities of each of the surgical article types that may be identified from the example writing surface 2210 of FIG. 34. A button 2308 is provided to confirm that the system accurately captured count information from the handwritten notations. A button 2310 is provided for the user to state that the count information gathered by the camera device recognizing handwritten notations is not accurate, and that the user is to enter corrections to the handwritten annotations. Other control inputs may be provided, for example, to disable or selectively enable use of the camera device and the text recognition software.

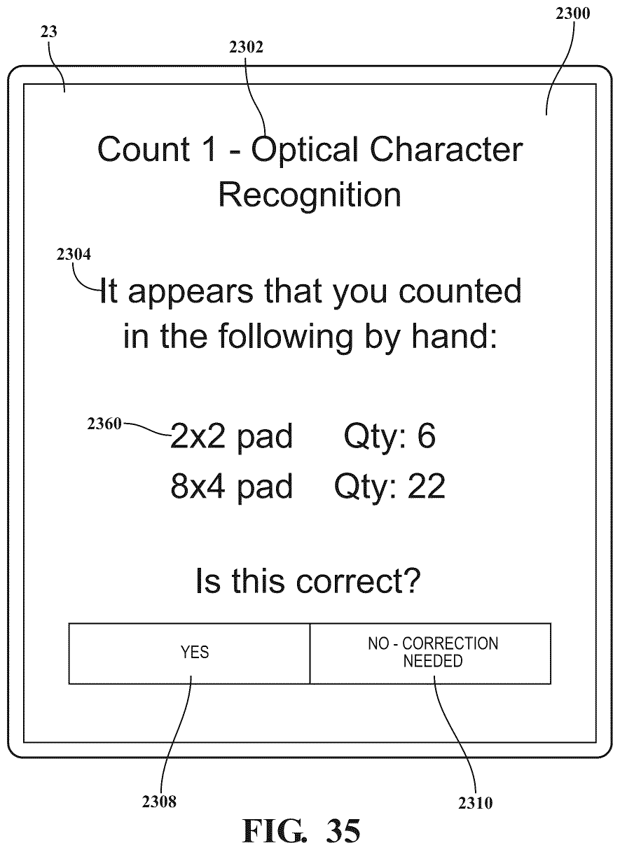

FIGS. 36A, 36B, and 36C illustrate a handwritten count in performed during a surgical procedure and representing a cumulative tally which may accrue over a period of time by the capturing and interpreting of a sequence of camera images of the handwritten count. FIG. 36A illustrates a writing surface 2210 including a plurality of surgical article types described in a first vertical column of the writing surface 2210 along a left side of the writing surface 2210. The writing surface 2210 is further illustrated including a plurality of boxes on a right side of the writing surface 2210, one corresponding to each of the surgical article types. FIG. 36A illustrates the writing surface 2210 after a first counting notation 2245 is made. FIG. 36B illustrates the same writing surface 2210 of FIG. 36A later in time, wherein a second counting notation 2245 is made next to the first counting notation 2245. FIG. 36C illustrates the same writing surface 2210 of FIG. 36B later in time, wherein a third counting notation 2245 is made next to the second counting notation 2245. By capturing and analyzing iterations of images of the writing surface 2210, the system may interpret use of surgical articles over time as they are counted in to the surgical procedure.

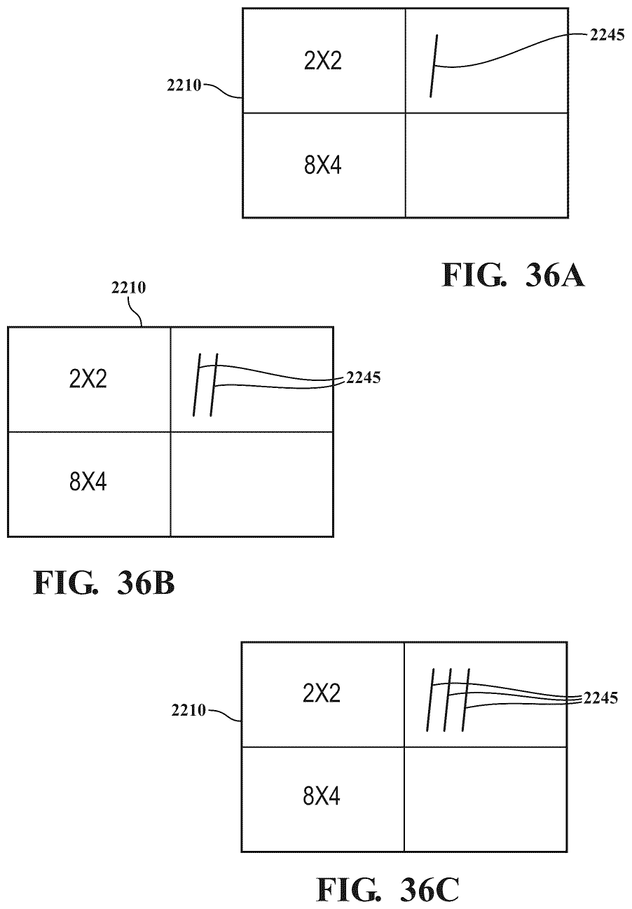

FIG. 37 is a flowchart illustrating a method 2400 for converting a handwritten count in into machine-coded text and machine-coded values for use in creating an electronic count based upon text recognition of the handwritten count. The method 2400 is provided capable of being operated with or upon system 5 of FIG. 1A, although it is intended that the method 2400 may be operated on other systems with other physical features and components. The method 2400 starts at step 2402. At step 2404, the user performs a manual, handwritten count upon an exemplary tally sheet or whiteboard. At step 2406, a camera device is utilized to capture an image of the manual, handwritten count. At step 2408, a determination is made whether the handwritten counting is complete. If the handwritten counting is not complete, the method 2400 returns to the step 2404. If the handwritten counting is complete, the method 2400 advances to step 2410, wherein exemplary optical character recognition (OCR) software is used to recognize characters from the manual, handwritten count. At step 2412, recognized characters related to surgical article types are converted into machine-encoded text and correlated with predefined article types, such as from a library of surgical article types stored in a computerized database. At step 2414, recognized characters related to quantity are converted into machine-coded values and associated with the machine-coded text to determine an electronic count.

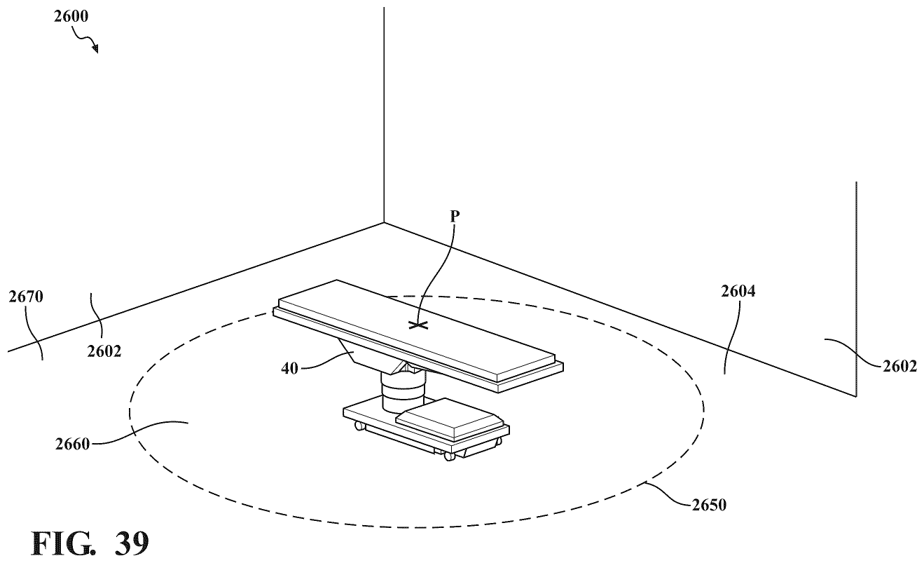

FIG. 40 schematically illustrates an exemplary computerized device 2710 including a neural network and configured to recognize handwritten notations related to a count of surgical articles, wherein the computerized device is configured to operate a machine learning algorithm configured to adapt and iterative improve recognition of data. Training inputs 2720 representative of handwritten notations that a user participating in a surgical procedure may make as part of a count in and/or count out process are provided to the computerized device 2710. The machine learning algorithm of the computerized device 2710 processes the training inputs 2720 and provides training output 2730 representing recognized text from the training inputs 2720. Corrective feedback 2740 is provided to the computerized device 2710, which the machine learning algorithm utilizes to improve operation of the algorithm and adjustable parameters therein, such that over a plurality of training cycles, the computerized device 2710 is capable of providing more accurate or excellent results.

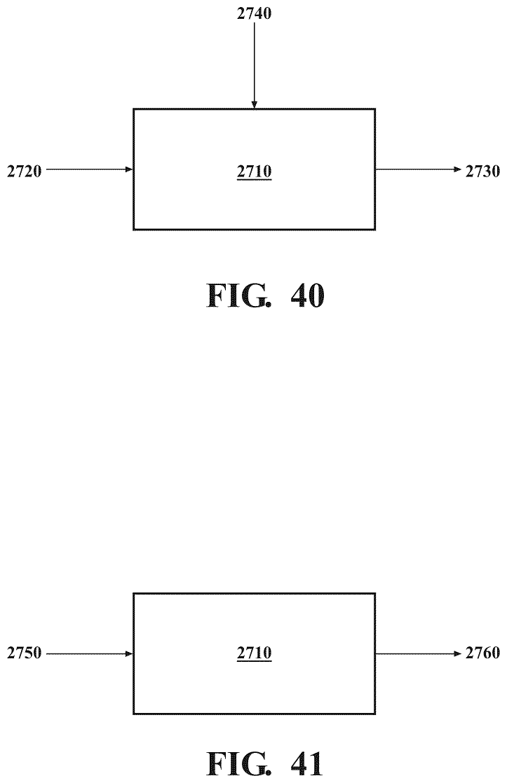

FIG. 41 schematically illustrates the computerized device 2710 processing operational data after training of the device 2710 is complete. Operational data 2750 including handwritten notations taken during a count in and/or count out process are provided to the computerized device 2710. The machine learning algorithm thereof, having been trained as described in relation to FIG. 40, provides outputs 2760 that may be used to generate an electronic count in accordance with the present disclosure.

IV—Monitoring Medical Accessory Location and movement

A vision system or camera device may be used to monitor visual information within an operating room or environment. For example, a medical professional may be determined through analysis of a series of captured images to be holding a scalpel in his or her hand. Location of that medical professional and/or the detected scalpel may be monitored, analyzed, and registered to a location within the operating room, for example, as an approximate distance from the camera unit 170 or the operating table 40 of FIG. 1A. In another example, an RF tag may be monitored by the navigation system 150 of FIG. 1A, and a location of the associated medical accessory may be monitored. A medical professional or programming of the system 130 may define a sterile field around the operating table 40. Locations of medical accessories may be compared to the sterile field, and an alert may be generated when the medical accessory is close to exiting or exits the sterile field. In a determined condition wherein a medical accessory is determined to have been outside the sterile field, a reaction may be taken, for example, providing an audible warning and requiring that the potentially contaminated medical accessory be counted out prior to the procedure being enabled to proceed.

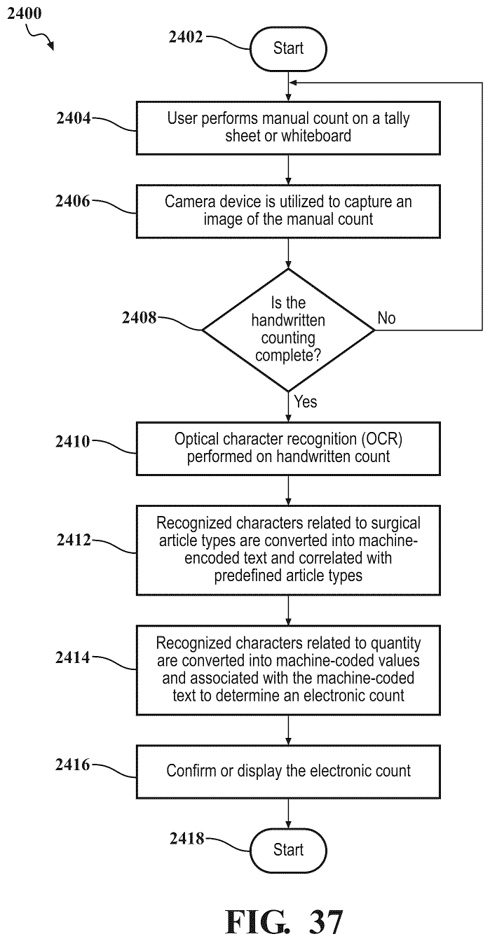

FIG. 38 illustrates an example of an operating room 2500 including walls 2502, a floor 2504, and an operating table 40. A first sterile field 2530A is defined upon the floor 2504 by a physical, visible boundary 2510 marked upon the floor 2504. The boundary 2510 may be created with paint, adhesive strips, reflective material, or other floor marking materials. The surgical system 5 of FIG. 1A may utilize the camera device 29 and/or other sensors to determine the location of the boundary 2510 upon the floor 2504 and make determinations based upon collected data whether a surgical accessory such as a sponge or surgical drape moves outside of the boundary 2510 and the associated first sterile field 2530A to an outside zone 2540A defined upon the floor 2504. Such movement may cause the surgical system 5 to issue a warning, an audible alert, a display upon display devices, or generate other similar output describing that the accessory has exited the first sterile field 2530A.

FIG. 38 further illustrates a second sterile field 2530B defined upon the floor 2504 by a virtual boundary 2520 associated with the floor 2504. A computerized processor within the surgical system 5 may operate programming which may be described as “geofencing”, defining the virtual boundary 2520 and monitoring/determining based upon collected data whether a surgical accessory such as a sponge or surgical drape moves outside of the virtual boundary 2520 and the associated second sterile field 2530B to an outside zone 2540B defined upon the floor 2504. Such movement may cause the surgical system 5 to issue a warning, an audible alert, a display upon display devices, or generate other similar output describing that the accessory has exited the second sterile field 2530B. The virtual boundary 2520 may be configurable, for example, with a medical professional being provided with a display representation of floor 2504 and with a moveable and resizable/shape-selectable box being superimposed upon the representation of the floor 2504 being capable of configuring the dimensions of the virtual boundary 2520 and the corresponding second sterile field 2530B. In one embodiment, the first sterile field 2530A and the second sterile field 2530B may be used in cooperation with each other. For example, with the first sterile field 2530A and the second sterile field 2530B being coincident, and the potentially redundant definition of the sterile field 2530A, 2530B reinforcing/increasing certainty of the location of the sterile field 2530A, 2530B. In another example, one of the sterile fields 2530A, 2530B may be larger than the other, with the smaller of the sterile field 2530A, 2530B being configured to generate a warning if a surgical accessory moves outside of the field, and with the second, larger of the sterile fields 2530A, 2530B generating a requirement that the surgical accessory be discarded and not used in the procedure for going outside of the larger field.

In another embodiment, the virtual boundary 2520 may be preconfigured to a fixed or predefined operating room 2500, with details of the room, procedures of the facility and medical professionals, and specific details of the operating room 2500 being taken into account in the defining of the virtual boundary 2520.

In one embodiment, a redline boundary upon the floor of the operating room 2500 may be used to create a virtual field or a geofence within the operating room 2500. The medical professionals, through operation of the surgical system 5, may be trained to look for the redline boundary. Such a virtual field or geofence may be automatically created by the redline. The virtual field may be configurable or reconfigurable by medical professionals, for example, enabling one of the medical professionals to draw a line on an input device screen or define a radius around the operating table 40. The surgical system 5 may reference a surgery schedule published for the facility and populate information regarding what the procedure being performed is going to be. The type of procedure may be used to dictate or predefine a size and type of sterile field used.

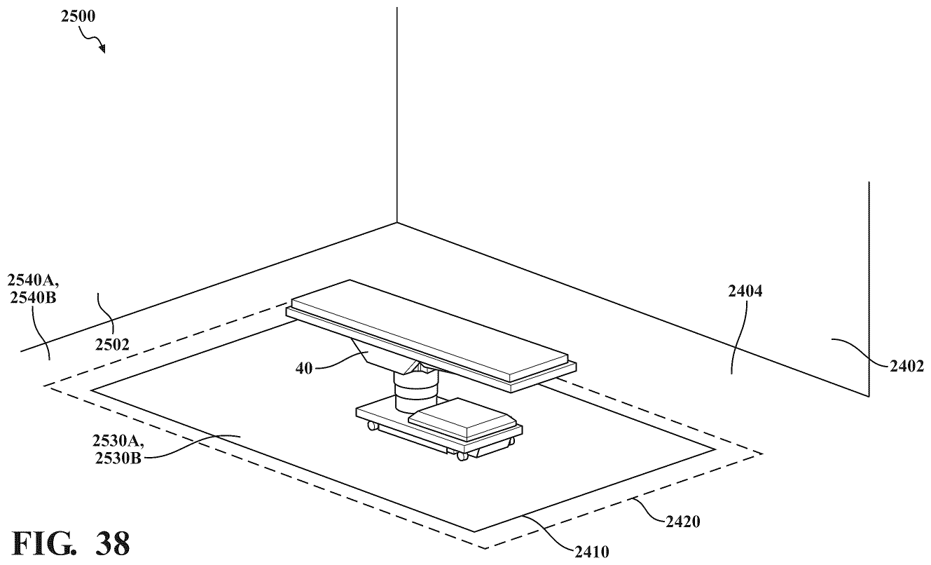

FIG. 39 is a perspective view of a second example of an operating room with an alternative sterile field defined by a distance from a center of an operating table. FIG. 39 illustrates the operating room 2600 with an alternative sterile field 2660 being defined by a distance from an object within the operating room 2600. In one embodiment, distances from an object within the operating room 2600 may be determined in three dimensions. In another embodiment, data may be rendered by a processor to consider a two-dimensional overview or floor plan of the floor 2604, with distances from the object within the operating room 2600 being determined as floor plan/two-dimensional horizontal distance from the object. In the example of FIG. 39, an object within the operating room 2600 includes a theoretical center P of the operating table 40. A constant distance from the center P is utilized by a computerized processor of the surgical system 5 of FIG. 1A to define a circular boundary 2650 to define the sterile field 2660 therewithin as distinct from a zone 2670 outside of the sterile filed 2660. In an alternative embodiment, the computerized processor may be programmed to determine a constant distance from an outer perimeter of the operating table 40 and define the sterile field 2660 according to that constant distance from around the operating table 40, which would result in the embodiment of FIG. 39 in a racetrack-shaped or rounded rectangle-shaped sterile filed 2660. A computerized processor within the surgical system 5 may operate geofencing programming, defining the boundary 2650 and monitoring/determining based upon collected data whether a surgical accessory such as a sponge or surgical drape moves outside of the virtual boundary 2650 and the associated sterile field 2660. Such movement may cause the surgical system 5 to issue a warning, an audible alert, a display upon display devices, or generate other similar output describing that the accessory has exited the sterile field 2660.

In some embodiments, a sponge count out procedure may require the sponge being counted out verbally to be moved additionally outside of a sterile field to complete the count out process, for example, as a two-part confirmatory process.

### V—Other Enabled Features

The aforementioned system and method may be utilized to confirm or adjust parameters of the medical procedure being performed. For example, a particular surgeon may enter preferences for a procedure upon a “preference card.” Such preferences may include operating parameters of equipment, particular medications to be used, particular sponge sizes to be used in the procedure, etc. The surgeon may alter or adjust preferences to his or her preference card through audible, visual, and/or RF inputs to the system. A medical professional may have a default preference card that is scanned into the system prior to the initiation of the procedure. Based upon changing conditions, pre-operative determinations, etc., the medical professional may scan an alternative preference card to change the parameters of the surgical procedure stored by the system. The system may perform a check and confirm or correct any parameters as being within or outside of recommended or safe parameter ranges.

Counting methods are disclosed herein to include manual counting, audible counting, counting based upon visual recognition based upon data captured by a camera device, and information provided through scanning of RF tags. The surgical system 5 of FIG. 1A may be configured to receive or request inputs from multiple sources in parallel. For example, a manual process, an audible count process, and an RF tag scanning process may be operated in parallel, with prompts from the surgical system 5 requesting the medical professional to enter each of the three inputs for each medical accessory counted in to the procedure and again for each medical accessory counted out of the procedure. Such a redundant system makes each operation or increment of the counting process a confirmed, intentional act and may provide for excellent accountability.

The microphones 60A, 60B of FIG. 1A may be utilized to configure or prompt routines to be operated by the surgical system 5. For example, a pre-operative “timeout” may be initiated and automated by microphone input. Such microphone inputs may be notarized and timestamped, for example, for later audit and process improvement. A variety of timers may be initiated and timestamped by the surgical system 5, for example, including the timeout before the procedure starts, surgical cite preparation procedures, an alcohol/surgical drape evaporation timer, a starting surgical site preparation timer, an intra-procedure timeout for checking details/calming medical professionals timer, etc.

In accordance with the disclosure, the surgical system 5 may scan or check a UDI of an object or surgical accessory. Such a UDI may be published or displayed upon packaging of an object or accessory. For example, a surgical instrument may be provided in sterile packaging. The medical professional may be prompted to scan or image the packaging of the surgical instrument prior to removing the instrument from the packaging. Such a method or process may prevent the professional from using the instrument without counting it in and may further save time in the procedure by avoiding having a medical professional from manually having to type in or scan through menus to select the instrument. Information accessed by the scanning of the UDI may be accessed from a variety of sources, for example, from a database or from a cloud resource or a remote server operated, for example, by a manufacturer of the device or by the facility operating the surgical system 5. Once accessed, such information may be made available, for example, by pushing the data to the tablet of the surgical sponge management system 10.

Instruments to be used in the procedure that are scanned in, via the UDI, are associated with the patient and the patient procedure. Such correlation of the instrument to the procedure may be used to provide excellent recordkeeping, training, error-proofing, inventory management, etc.

Because the instruments are scanned in, target parameters and the instructions for use (IFU) may be displayed or made readily accessible to medical professionals conducting the procedure. The UDI and corresponding inputs received through the microphone 190 may be utilized in cooperation, for example, as part of timeout process, double checking procedure details and instruments to be used prior to a portion of the procedure being cleared to start. Additionally, UDI and corresponding audible inputs may be used to prompt or initiate showing a picture of the surgical site on the dashboard display, for example, for training or supervisory purposes.

The counting in process along with the scanning of the UDI from each object to be utilized in the procedure may have excellent benefits related to managing medical procedure details. For example, a particular procedure may be preconfigured to require three of a first object and two of a second object. As the objects are counted in to the medical procedure, the surgical system 5 may publish or announce details of the counted in objects versus the preconfigured list of objects expected to be needed in the procedure. The surgical system 5 may distinguish between voices of registered medical professionals, for example, requiring a particular count in or count out instance to be performed by the registered surgeon or by the registered circulating nurse assigned to the procedure based upon voice recognition being able to confirm the identity of the speaker. In another instance, the circulating nurse and a scrub technician may both be required to verbally confirm an action or checklist item, with the voices of the particular professional being required to complete confirmation.

The various inputs and sensors of the surgical system 5 may be utilized to comprehensively check milestones in a predetermined milestone list for a procedure. Such a method or process may include two-factor confirmation that each milestone is certifiably passed, and such milestones may be documented and timestamped.

### VI—Clauses

Clause 1: A computer-implemented method of facilitating a surgical procedure with a surgical management system including an imaging device, a display, a user interface, and one or more processors is provided. The method includes capturing, with the imaging device, an image of a sponge reconciliation board including a list of surgical article types and a handwritten count of a quantity of each of the surgical article types that have been counted in to the surgical procedure. The method further includes converting, with the one or more processors, the handwritten counts into machine-encoded values, converting, with the one or more processors, the list into machine-encoded texts of the surgical article types, and associating, with the one or more processors, each of the machine-encoded values with one of the machine-encoded texts of the surgical article types. The method further includes correlating, with the one or more processors, the machine-encoded texts with predefined electronic article types stored in a database, displaying, on the display, an electronic count of the quantity and associated surgical article type for a plurality of surgical articles that have been counted in to the surgical procedure, and receiving, on the user interface, one or more subsequent inputs to alter the electronic count.
Clause 2: In some embodiments, the electronic count is a first count of the surgical articles that have been counted in to the surgical procedure, and the one or more subsequent inputs on the user interface includes a second manual count of the surgical articles that have been counted in to the surgical procedure.
Clause 3: In some embodiments, the method further includes displaying, on the display, the one or more subsequent inputs as a correction made to the electronic count.
Clause 4; In some embodiments, the user interface is a computerized tablet device, and displaying the electronic count on the display includes displaying the electronic count on the computerized tablet device such that the second manual count is entered upon the computerized tablet device upon which the electronic count is being displayed.
Clause 5: In some embodiments, the electronic count is a first count of the surgical articles that have been counted in to the surgical procedure, the one or subsequent inputs on the user interface to alter the electronic count includes a second count independent from the electronic count, and the second count alters the electronic count by correcting inaccuracies in the electronic count.
Clause 6: In some embodiments, the second count includes implementation of manual entry to the user interface, use of unique device identifier (UDI) code in combination with a UDI scanning device, receiving through a microphone a verbal count and processing the verbal count with voice recognition software, use of a camera device to implement visual recognition of the surgical articles, or use of a plurality of radio frequency (RF) identification tags and a RF receiver device to implement recognition of the surgical articles.
Clause 7: In some embodiments, the method further includes utilizing a camera device to monitor and determine occurrence of a body cavity search during the surgical procedure configured to confirm that the surgical articles have been entirely removed from a body cavity.
Clause 8: In some embodiments, the electronic count is an initial count performed during a pre-surgical portion of the surgical procedure, and the one or subsequent inputs on the user interface includes a second subsequent count of surgical articles counted in after the initial count.
Clause 9: In some embodiments, the handwritten count on the sponge reconciliation board is performed by a first surgical professional, and wherein the one or more inputs on the user interface are performed by a second surgical professional.
Clause 10: A computer-implemented method of facilitating a surgical procedure with a surgical management system including an imaging device, a display, a user interface, and one or more processors is provided. The method includes, during a pre-surgical portion of the surgical procedure as part of a count in process, capturing, with the imaging device, a first image of a first sponge reconciliation board including a list of surgical article types and a handwritten count of a quantity of each of the surgical article types corresponding to a plurality of surgical articles that have been counted in to the surgical procedure. The method further includes, during the pre-surgical portion of the surgical procedure as part of the count in process, converting, with the one or more processors, the handwritten counts of the quantity of each of the surgical article types that have been counted in to the surgical procedure into a first set of machine-encoded values, converting, with the one or more processors, the list into machine-encoded texts of the surgical article types, and associating, with the one or more processors, each of the first set of machine-encoded values with one of the machine-encoded texts of the surgical article types. The method further includes, during the pre-surgical portion of the surgical procedure as part of the count in process, correlating, with the one or more processors, each of the machine-encoded texts with a predefined article type stored in a database and displaying, on the display, a first electronic count of the quantity and the correlated predefined article type for each of the plurality of surgical articles that have been counted in to the surgical procedure. The method further includes, during the surgical procedure as part of a count out process, capturing, with the imaging device, a second image of a sponge reconciliation board including the list of surgical article types and a handwritten count of a quantity of each of the surgical article types corresponding to the plurality of surgical articles that have been counted out of the surgical procedure. The method further includes, during the surgical procedure as part of the count out process, converting, with the one or more processors, the handwritten counts of the quantity of each of the surgical article types that have been counted out of the surgical procedure into a second set of machine-encoded values, associating, with the one or more processors, each of the second set of machine-encoded values with one of the machine-encoded texts of the surgical article types and the correlated one of the predefined article types, and displaying, on the display, a second electronic count of the quantity and the correlated predefined article type for each of the plurality of surgical articles that have been counted out of the surgical procedure. The method further includes receiving, on the user interface, one or more inputs to alter the first electronic count or the second electric count.
Clause 11: A computer-implemented method of facilitating a surgical procedure with a surgical management system including an imaging device, a display, a user interface, and one or more processors is provided. The method includes training a neural network by iteratively receiving one of a plurality of training images of a training sponge reconciliation board and recognizing textual information from the training sponge reconciliation board. Training the neural network further includes identifying from the textual information a list of a plurality of surgical article types, identifying from the textual information a handwritten count of a quantity of each of the plurality of surgical article types that have been counted in to the surgical procedure, and correlating each of the plurality of surgical article types with one of a plurality of predefined article types stored in a database. Training the neural network further includes generating an electronic count of the quantity and the correlated predefined article type based upon the received image and correcting the generated electronic count. The method further includes capturing, with the imaging device, an operative image of an operative sponge reconciliation board including a second list of at least two of the plurality of surgical article types and a handwritten count of a quantity of each of the plurality of surgical article types from the list that have been counted in to the surgical procedure. The method further includes, within the one or more processors, utilizing the trained neural network to process the operative image and generate the electronic count of the quantity and the correlated predefined article type based upon the operative image and displaying, on the display, the electronic count of the quantity and correlated predefined article type. The method further includes receiving, on the user interface, one or more subsequent inputs to alter the electronic count.
Clause 12: A computer-implemented method of facilitating a surgical procedure with a surgical management system including an imaging device, a display, a user interface, and one or more processors is provided. The method includes, during the surgical procedure, iteratively capturing, with the imaging device, one of a sequence of images of a sponge reconciliation board including a list of surgical article types and a handwritten count of a quantity of each of the surgical article types that are counted in over a course of the surgical procedure. The method further includes, for each of the sequence of images, converting, with the one or more processors, the handwritten counts of the quantity of each of the surgical article types that are counted in into a first set of machine-encoded values. The method further includes converting, with the one or more processors, the list into machine-encoded texts of the surgical article types, associating, with the one or more processors, each of the first set of machine-encoded values with one of the machine-encoded texts of the surgical article types, and correlating, with the one or more processors, the machine-encoded texts with a predefined article types stored in a database. The method further includes, after each of the sequence of images is captured, for each of the surgical article types, iteratively updating an electronic count of the quantity and the correlated predefined article type that have been counted in so far to the surgical procedure. The method further includes displaying, on the display, for each of the surgical article types, the updated electronic count of the quantity and the correlated predefined article type that have been counted in so far to the surgical procedure and receiving, on the user interface, one or more subsequent inputs to alter the electronic count.
Clause 13: In some embodiments, the method further includes, during the surgical procedure, iteratively capturing, with the imaging device, one of a second sequence of images of the sponge reconciliation board or a second sponge reconciliation board including the list of surgical article types and a handwritten count of a quantity of each of the surgical article types that are counted out over the course of the surgical procedure. The method further includes, for each of the second sequence of images, converting, with the one or more processors, the handwritten counts of the quantity of each of the surgical article types that are counted out into a second set of machine-encoded values, associating, with the one or more processors, each of the second set of machine-encoded values with one of the machine-encoded texts of the surgical article types, and, after each of the second sequence of images is captured, for each of the surgical article types, iteratively updating a second electronic count of the quantity and the correlated predefined article type that have been counted out so far of the surgical procedure. The method further includes displaying, on the display, for each of the surgical article types, the updated second electronic count of the quantity and the correlated predefined article type that have been counted out so far of the surgical procedure.
Clause 14: A method of facilitating a surgical procedure with a surgical management system including one or more processors is provided. The method includes receiving, through a camera device or a radio frequency (RF) receiver device, data indicating a location of a surgical accessory to be utilized in the surgical procedure, monitoring a zone of locations defined as a sterile field for the surgical procedure, and comparing the location of the surgical accessory to the zone of locations. The method further includes generating an output designating the surgical accessory as no longer being sterile based upon the comparing.
Clause 15: A method of facilitating a surgical procedure with a surgical management system including one or more processors is provided. The method includes receiving, through a camera device or a radio frequency (RF) receiver device, data indicating a location of a surgical accessory to be utilized in the surgical procedure and monitoring a location of interest for the surgical procedure. The method further includes defining an acceptable location zone within a defined range of the location of interest, comparing the location of the surgical accessory to the location of interest, and generating an output designating the surgical accessory as no longer being acceptable for use in the surgical procedure based upon the comparing indicating that the surgical accessory was moved outside of the acceptable location zone.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A computer-implemented method of facilitating a surgical procedure with a surgical management system including a display device, a user interface, one or more microphones, and one or more processors, the method comprising:
receiving, with the user interface, a first input including a first surgical article type and a first quantity of the first surgical article type for a plurality of surgical articles that have been counted in to the surgical procedure;
displaying, on the display, an electronic count including the first surgical article type and the first quantity of the first surgical article type;
receiving, on the user interface, one or more subsequent inputs to alter the electronic count;
displaying, on the display, the altered electronic count;
capturing, with the one or more microphones, audible speech including a confirming verbal count for the plurality of surgical articles that have been counted in to the surgical procedure;
converting, with the one or more processors, the confirming verbal count into machine-encoded values including a second surgical article type and a second quantity of the second surgical article type;
comparing, with the one or more processors, the altered electronic count to the machine-encoded values to identify a discrepancy between the altered electronic count and the machine-encoded values; and
displaying, upon the display device, the identified discrepancy between the altered electronic count and the machine-encoded values.

2. The computer-implemented method of claim 1, wherein comparing the altered electronic count to the machine-encoded values includes:
comparing the first surgical article type to the second surgical article type, and
comparing the first quantity of the first surgical article type to the second quantity of the second surgical article type.

3. The computer-implemented method of claim 1, further comprising:
receiving, with the user interface, a count out input including a counted-out quantity of the first surgical article type for a plurality of surgical articles that have been counted out of the surgical procedure;
updating the electronic count based upon the counted-out quantity of the first surgical article type; and
displaying, on the display, the updated electronic count of the first surgical article type and the first quantity of the first surgical article type.

4. The computer-implemented method of claim 1, wherein the first input further includes a first list of a plurality of surgical article types and a first count of a quantity of each of the plurality of surgical article types for the plurality of surgical articles that have been counted in to the surgical procedure;
wherein the electronic count further includes the first list of the plurality of surgical article types and the first count of the quantity of each of the plurality of surgical article types for the plurality of surgical articles that have been counted in to the surgical procedure; and
wherein the machine-encoded values further include a second list of the plurality of surgical article types and a second count of the quantity of each of the plurality of surgical article types that have been counted in to the surgical procedure.

5. The computer-implemented method of claim 4, further comprising:
receiving, with the user interface, a count out input including the first list of the plurality of surgical article types and a count of a counted-out quantity of each the plurality of surgical article types for a plurality of surgical articles that have been counted out of the surgical procedure;
updating the electronic count based upon the count out input; and
displaying, on the display, the updated electronic count.

6. The computer-implemented method of claim 1, further comprising prompting the confirming verbal count prior to authorizing a portion of the surgical procedure to commence.

7. The computer-implemented method of claim 1, wherein the confirming verbal count is a first confirming verbal count;
wherein the machine-encoded values are a first set of machine-encoded values; and
further comprising:
capturing, with the one or more microphones, additional audible speech including a second confirming verbal count for the plurality of surgical articles that have been counted in to the surgical procedure;
converting, with the one or more processors, the second confirming verbal count into a second set of machine-encoded values including a third surgical article type and a third quantity of the third surgical article type that have been counted in to the surgical procedure;
further comparing, with the one or more processors, the second set of machine-encoded values to the altered electronic count and to the machine-encoded values; and
displaying, upon the display device, a second discrepancy either between the second set of machine-encoded values and the altered electronic count or between the second set of machine-encoded values and the first set of machine-encoded values.

8. The computer-implemented method of claim 1, further comprising:
registering a user;
identifying the registered user as entering the one or more subsequent inputs; and
displaying, upon the display, an indication that the registered user entered the one or more subsequent inputs.

9. The computer-implemented method of claim 1, further comprising:
registering a plurality of users authorized to alter the electronic count;
receiving a plurality of subsequent inputs to alter the electronic count;
for each of the plurality of subsequent inputs, identifying one of the plurality of users as being a respective user that entered the subsequent input; and
displaying, on the display, for each of the plurality of subsequent inputs, an indication identifying the user that entered the subsequent input.

10. The computer-implemented method of claim 1, wherein the user interface is a touch screen device; and
further comprising, upon the touch screen device:
displaying the first surgical article type for a plurality of surgical articles that have been counted in to the surgical procedure;
displaying, adjacent to the displayed first surgical article type, a first button indicating an increase and a second button indicating a decrease;
monitoring a user indicating by touch to one of the first button or the second button; and
receiving the first input or one of the one or more subsequent inputs based upon the monitored indicating.

11. The computer-implemented method of claim 1, further comprising determining a number of users that entered the one or more subsequent inputs;
comparing the determined number of users that entered the subsequent inputs to a threshold number of users; and
generating an alert when the comparing indicates that the determined number of users that entered the subsequent inputs exceeds the threshold number of users, wherein the alert is configured to raise awareness that an increased risk of miscounting is indicated due to the determined number of users that entered the subsequent inputs.

12. A computer-implemented method of facilitating a surgical procedure with a surgical management system including a display device, a user interface, one or more microphones, and one or more processors, the method comprising:
receiving, with the user interface, a first input including a first list of a plurality of surgical article types and a first count of a quantity of each of the plurality of surgical article types for a plurality of surgical articles that have been counted in to the surgical procedure;
displaying, on the display, an electronic count of the first list of the plurality of surgical article types and the first count of the quantity of each of the plurality of surgical article types for the plurality of surgical articles that have been counted in to the surgical procedure;
receiving, on the user interface, one or more subsequent inputs to alter the electronic count;
displaying, on the display, the altered electronic count;
capturing, with the one or more microphones, audible speech including a confirming verbal count for the plurality of surgical articles that have been counted in to the surgical procedure;
converting, with the one or more processors, the confirming verbal count into machine-encoded values including a second list of the plurality of surgical article types and a second count of the quantity of each of the plurality of surgical article types that have been counted in to the surgical procedure;
comparing, with the one or more processors, the altered electronic count to the machine-encoded values to identify a discrepancy between the altered electronic count and the machine-encoded values; and
displaying, upon the display device, the identified discrepancy between the altered electronic count and the machine-encoded values.

13. The computer-implemented method of claim 12, further comprising:
receiving, with the user interface, a count out input including the first list of the plurality of surgical article types and a count of a counted-out quantity of each the plurality of surgical article types for a plurality of surgical articles that have been counted out of the surgical procedure;
updating the electronic count based upon the count out input; and
displaying, on the display, the updated electronic count.

14. The computer-implemented method of claim 12, wherein comparing the altered electronic count to the machine-encoded values includes:
comparing the first list of the plurality of surgical article types to the second list of the plurality of surgical article types, and
comparing the first count of the quantity of each of the plurality of surgical article types to the second count of the quantity of each of the plurality of surgical article types.

15. The computer-implemented method of claim 12, further comprising prompting the confirming verbal count prior to authorizing a portion of the surgical procedure to commence.

16. The computer-implemented method of claim 12, further comprising:
registering a user;
identifying the registered user as entering the one or more subsequent inputs; and
displaying, upon the display, an indication that the registered user entered the one or more subsequent inputs.

17. A computer-implemented method of facilitating a surgical procedure with a surgical management system including a display device, a user interface, one or more microphones, and one or more processors, the method comprising:
within the one or more processors, training a neural network by iteratively:
receiving an auditory input including one of a plurality of training verbal inputs including a training list of a plurality of surgical article types and a training count of a quantity of each of the plurality of surgical article types, the plurality of training verbal counts being configured to simulate a user counting in a plurality of surgical articles into the surgical procedure;
recognizing data related to one of the plurality of surgical article types or the quantity of each of the plurality of surgical article types from the received one of the plurality of training verbal inputs;
based upon the recognized data, generating a training list result of the plurality of surgical article types and a training quantity result of each of the plurality of surgical articles types;
scoring the training list result and the training quantity result based upon details of the one of the plurality of training verbal inputs; and
correcting a next iteration of recognizing the data based upon the scoring;
receiving, with the user interface, a first input including a first list of a plurality of surgical article types and a first count of a quantity of each of the plurality of surgical article types for a plurality of surgical articles that have been counted in to the surgical procedure;
displaying, on the display, an electronic count of the first list of the plurality of surgical article types and the first count of the quantity of each of the plurality of surgical article types for the plurality of surgical articles that have been counted in to the surgical procedure;
receiving, on the user interface, one or more subsequent inputs to alter the electronic count;
displaying, on the display, the altered electronic count;
capturing, with the one or more microphones, audible speech including a confirming verbal count for the plurality of surgical articles that have been counted in to the surgical procedure;
utilizing, within the one or more processors, the trained neural network to process the audible speech and generate a recognized list of the plurality of surgical article types and a recognized count of the quantity of each of the plurality of surgical article types;
comparing, with the one or more processors, the altered electronic count to the recognized list and the recognized count to identify a discrepancy between the confirming verbal count and the altered electronic count; and
displaying, upon the display device, the identified discrepancy.

18. The computer-implemented method of claim 17, further comprising:
receiving, with the user interface, a count out input including the first list of the plurality of surgical article types and a count of a counted-out quantity of each the plurality of surgical article types for a plurality of surgical articles that have been counted out of the surgical procedure;
updating the electronic count based upon the count out input; and
displaying, on the display, the updated electronic count.

19. The computer-implemented method of claim 17, further comprising prompting the confirming verbal count prior to authorizing a portion of the surgical procedure to commence.

20. The computer-implemented method of claim 17, further comprising:
registering a user;
identifying the registered user as entering the one or more subsequent inputs; and
displaying, upon the display, an indication that the registered user entered the one or more subsequent inputs.
